(19) United States
(12) Patent Application Publication
(10) Pub. No.: US 2012/0304318 A1
(43) Pub. Date: Nov. 29, 2012

(54) CANCER DIAGNOSIS AND TREATMENT

(75) Inventors: Shin-ichi Ohnuma, London (GB); John Daniel Kelly, London (GB); Ryuji Hamamoto, Tokyo (JP); Julie Watson, Cambridge (GB)

(73) Assignees: CAMBRIDGE ENTERPRISE LTD, Cambridge (GB); UCL BUSINESS PLC, London (GB)

(21) Appl. No.: 13/516,749

(22) PCT Filed: Dec. 17, 2010
(86) PCT No.: PCT/GB10/02294

§ 371 Date: Jun. 18, 2012

(30) Foreign Application Priority Data
Dec. 17, 2009 (EP) 0922085.6

(51) Int. Cl. A61K 31/7088; C12Q 1/68; G01N 33/574; G01N 33/566; A61P 35/00; C12N 15/85; A01K 67/00; A61K 49/00; A61K 31/407; C40B 30/04; C12N 15/63

(52) U.S. Cl. 800/3; 506/9; 435/6.12; 435/6.11; 435/7.1; 436/501; 435/471; 435/455; 435/6.13; 800/10; 514/44 R; 514/410

(57) ABSTRACT

The invention concerns materials and methods relating to the use of OMD (osteomodulin) and\or PRELP (Proline/arginine-rich end leucine-rich repeat protein) expression, particularly under-expression, to discriminate cancer and non-cancer cells in a variety of cancers. The invention further provides methods and materials based on OMD and\or PRELP for use in therapy e.g. to suppress cancer initiation or development.

### TECHNICAL FIELD

The present invention relates generally to methods and materials for use in treatment and diagnosis of cancers such as and other cancers, for example bladder, kidney, lung, breast, stomach, colon, rectum, prostate, utrine cervix, endometrium, ovary, thyroid grand, esophagus, small intestine, and adrenal gland cancers.

### BACKGROUND ART

Cancer is a disease in which cells display un-controlled anchorage independent growth resulting in disruption of tissue homeostasis. Thus, after initiation of cancer at the original location, they spread to other locations in the body through metastasis and invasion. Since cancer is caused by a variety of gene alternations, there is no general method for treatment. Recently, significant advances in cancer treatment have been achieved. However, many cancers still do not respond to treatment, and many still prove fatal. Many oncogenes and tumour suppressor genes have been identified, and many methods of diagnosis have been developed based on these genes. However, methods of diagnosis still remain inadequate and this development is also far from satisfactory. Development of general diagnosis of a majority of cancer at early stages is very important.

Bladder cancer and kidney cancer are major types of urological tumour. A majority of bladder cancer patients have non-muscle invasive bladder cancer, stage pTa or pT1, with a good prognosis. However, bladder cancer has the highest recurrence rate of any solid tumour, and 60-70% patients will develop a recurrence. Ca.10% of these recurrences will progress to advanced muscle invasive tumour. Therefore, early detection and determination of the precise stages of bladder cancer is required.

Early detection of bladder cancer and its recurrences is essential for improved prognosis and long-term survival. Several tests for bladder cancer have been reported including the urinary bladder cancer test and the lewis X antigen test. However, their sensitivity and specificity are largely in the range of 50-70%. Some diagnosis methods have high sensitivity but low specificity, while others have high specificity and low sensitivity. For example, FISH has 30% sensitivity and 95% specificity (Gudjonsson et al., 2008), while HA-HAase has 86% sensitivity and 61% specificity (Eissa et al., 2005). There is no perfect method to identify cancer tissue with high accuracy. This situation is also true of kidney cancer. Kidney cancer is another type of urological cancer. The two most common types of kidney cancer are renal cell carcinoma and renal pelvis carcinoma. Around 200,000 new cases of kidney cancer are diagnosed in the world each year. In the UK, kidney cancer is the eighth most common cancer in men. The highest rates are recorded in North America. However, still there is no ideal diagnosis method. Therefore, it is very important to develop a method that discriminates cancer and non-cancer bladder/kidney cells with high sensitivity and specificity.

It will be appreciated from the forgoing that the provision of newly characterised, specific, reliable markers that are differentially expressed in normal and transformed tissues would provide a useful contribution to the art. Markers which appear to be “universal markers” (i.e. associated with many different types of cancer) are particularly useful since they can be used to reduce the cost and time of diagnosis. Any such markers could be used inter alia in the diagnosis of cancers such as bladder/kidney cancer, the prediction of the onset of cancers such as bladder/kidney cancer, or the treatment of cancers such as bladder/kidney cancer.

### DISCLOSURE OF THE INVENTION

The present inventors have shown that the expression of two proteins, OMD (osteomodulin, also known as osteoadherin) and PRELP (Proline/arginine-rich end leucine-rich repeat protein) may be used to discriminate cancer and non-cancer bladder/kidney cells with high sensitivity and specificity. Furthermore, the combination of OMD and PRELP expression analysis provides even greater accuracy in the determination of both bladder cancer and kidney cancer. They have also shown that the proteins can be used to discriminate cancer and non-cancer cells in other cancers such as lung, breast, stomach, colon, rectum, prostate, utrine cervix, endometrium, ovary, thyroid grand, esophagus, small intestine, and adrenal gland cancers.

Nature. 2000 Aug. 17; 406(6797):747-52 “Molecular portraits of human breast tumours”. Perou et al reports a reduction of expression of OMD mRNA in Estrogen receptor positive vs negative cancers. However the same paper refers to an increase in OMD mRNA in Normal Fibroadenoma vs Invasive Lobular Carcinoma.

OMD or PRELP have previously been referred to in various published patent applications in the technical field of cancer:

WO2008104543A2 (EP1961825) describes OMD as being a “bone metastasis associated gene” and apparently observed that metastatic breast cancer cells localized in bone consistently showed a strong immunoreactivity to OMD in the majority of the samples analyzed. PRELP is also referred to.

WO04108896A2 relates to gene expression profiling of uterine serous papillary carcinomas and ovarian serous papillary tumors. It notes that these are histologically indistinguishable and seeks to find whether oligonucleotide microarrays may differentiate them, Down regulation of OMD is referred to in the context of uterine serous papillary carcinoma.

WO2008077165A1 is concerned with the need for reliable and efficient breast cancer diagnostic and prognostic methods and means. It describes a set of moieties specific for at least 200 tumor markers which include OMD.

EP2028492A1 is concerned with the provision of tumor markers which are highly specific to colon cancer and with the provision of a method capable of identifying the morbidity of colon cancer. PRELP is referred to as being a colon-cancer related protein which is down-regulated.

The utility of OMD and PRELP in the presently claimed invention is not taught in these documents.

OMD and PRELP make a sub-branch in the phylogenetic tree (FIG. 1). Their structure, expression, and function are different from members in other sub-branches of the small leucine-rich repeat proteoglycans (SLRP) family. However there has previously been little investigation into the role of OMD and PRELP in cancers, and in particular urological cancers.

In addition to the disclosure herein that expression of the genes encoding OMD and PRELP is an ideal method for the diagnosis of cancer, it is further disclosed that activation of OMD or/and PRELP gene expression or function can suppress cancer initiation and development.

Accordingly, the present invention describes the use of OMD and PRELP (either of which may be referred to hereinafter as a “target protein” of the present invention”) as markers of cancer, and provides methods for their use in such applications.

As discussed in detail below, the target proteins of the present invention are of particular use inter alia as diagnostic and prognostic markers of a variety of cancers, and in particular epithelial cancers and bladder or kidney cancers. As with known markers, they may be used for example to assist diagnosing the presence of cancer at an early stage in the progression of the disease and predicting the likelihood of clinically successful outcome, particularly with regard to the sensitivity or resistance of a particular patient's tumour to a chemotherapeutic agent or combinations of chemotherapeutic agents. Furthermore these targets can be used for therapeutic intervention in bladder or kidney and other cancers e.g. to specifically target neoplastic cells without causing significant toxicity in healthy tissues, and to provide methods for the evaluation of the ability of candidate therapeutic compounds to modulate the biological activity of cancerous cells from the bladder or kidney and other tissues.

Thus the present invention relates to the diagnosis and treatment of cancer, and specifically to the discrimination of neoplastic cells from normal cells on the basis of under-expression of specific tumour antigens and the targeting of treatment through exploitation of the differential expression of these antigens within neoplastic cells. The invention specifically relates to the detection of one or more proteins (”target proteins“) that are under-expressed in neoplastic cells compared with the expression in pathologically normal cells (see e.g. Tables 2 to 4). Accordingly, these target proteins, as well as nucleic acid sequences encoding them, or sequences complementary thereto, can be used as cancer markers useful in diagnosing or predicting the onset of a cancer such as bladder or kidney cancer, monitoring the efficacy of a cancer therapy and/or as a target of such a therapy.

The invention in particular relates to the discrimination of neoplastic cells from normal cells on the basis of the under-expression of a target protein of the present invention, or the gene that encodes this protein. To enable this identification, the invention provides a pattern of expression of a specific protein, the expression of which is decreased in neoplastic cells in comparison to normal cells. The invention provides a variety of methods for detecting this protein and the expression pattern of this protein and using this information for the diagnosis or prognosis and treatment of cancer, or assessment of efficacy of cancer treatments.

For example such methods may include:
Detection or measurement of mRNA of OMD and/or PRELP in samples from an individual and correlation of the levels detected with the likelihood, stage or susceptibility of cancer (e.g. an epithelial cancer, such as a urological cancer like bladder or kidney cancer) in that individual;
Detection or measurement of suppression of transcription or translation of OMD and/or PRELP in samples from an individual and correlation of the levels detected with the likelihood, stage or susceptibility of cancer (e.g. an epithelial cancer, such as a urological cancer like bladder or kidney cancer) in that individual;
Detection or measurement of protein levels of OMD and/or PRELP in samples from an individual and correlation of the levels detected with the likelihood, stage or susceptibility of cancer (e.g. an epithelial cancer, such as a urological cancer like bladder or kidney cancer) in that individual;
Detection or measurement of OMD and/or PRELP activity in samples from an individual and correlation of the levels detected with the likelihood, stage or susceptibility of cancer (e.g. an epithelial cancer, such as a urological cancer like bladder or kidney cancer).

Furthermore, in other aspects the invention provides novel screening systems and therapeutics for treating cancers such as bladder or kidney cancer which include those which:
Increase the activity of OMD and/or PRELP e.g. by stabilisation of the proteins, or other modification
Increase the expression of genes encoding OMD and/or PRELP e.g. by transcriptional activation of the genes, or introduction of nucleic acid encoding the proteins
Comprise variants or analogues that have an activity similar to of OMD and/or PRELP

The present invention thereby provides a wide range of novel methods for the diagnosis, prognosis and treatment of cancers, including bladder or kidney cancer, on the basis of the differential expression of the target proteins. These and other numerous additional aspects and advantages of the invention will become apparent to the skilled artisan upon consideration of the following detailed description of the invention.

### DETAILED DESCRIPTION OF THE PRESENT INVENTION

As described herein, we have found that the expression levels of OMD and PRELP are significantly down-regulated in many cancers. In aspects and embodiments described herein the cancer may be an epithelial cancer e.g. a urological cancer such as bladder and renal cell carcinoma. In other aspects or embodiments it may be a lung, breast, stomach, colon, rectum, prostate, utrine cervix, endometrium, ovary, thyroid grand, esophagus, small intestine, or adrenal gland cancer,

Certain preferred protein\cancer combinations embraced by the invention include OMD\lung cancer; PRELP\lung cancer; PRELP\Prostate cancer; PRELP\breast cancer; and so on.

We could clearly distinguish tumor from normal when combining gene expression data for both genes, even in very early stages of carcinogenesis. These novel findings provide for novel methods of cancer diagnosis. Also, activation of these genes potentiates apoptosis of cancer cells without influencing normal cells and sensitises to cancer drugs, demonstrating that activation of these genes can provide a novel treatment of cancer.

Furthermore, a xenograft study using cancer cells showed that in vivo overexpression in mice completely suppressed development of cancer.

Some of these aspects and embodiments will now be described in more detail:

OMD

OMD is a keratan sulphate proteoglycan belonging to the SLRP family (Sommarin et al., 1998). OMD has a high affinity for hydroxyapatite, which is a unique feature among the SLRPs probably mediated by the extended C-terminal region that consists of roughly 60% acidic residues. OMD is expressed from early differentiated osteoblasts and peaks late in osteoid formation and at the start of mineral deposition and has been proposed as an organizer of the ECM. OMD is regulated by TGF-β1 and BMP-2, and is a marker for early terminally differentiated osteoblasts (Rehn et al., 2006).

As disclosed herein, the expression of OMD can be significantly reduced in many types of malignant cancers including bladder and renal carcinomas compared to normal tissue.

Bladder cancer is characterized by frequent genetic alterations of chromosome 9 and the OMD gene is located at chromosome 9q22.31. Refined deletion mapping with microsatellite markers has suggested the existence of several putative tumor suppressor loci on this chromosome at 9p22-23, 9p21-22, 9p11-13, 9q12-13, 9q21-22, 9q31 and 9q33-34 (Czerniak et al., 1999; Habuchi et al., 1995; Simoneau et al., 1996; Simoneau et al., 1999). In the light of the results herein, we show a significant deletion of the OMD gene locus in malignant bladder tissues using a 1 Mb CGH array (data not shown).

PRELP

PRELP was originally identified as an abundant protein within the extracellular matrix (ECM) of cartilage (Heinegard et al., 1986), and was also detected at lower levels in other connective tissues where it has been localized close to the BM (Stanford et al., 1995). PRELP was postulated to interact with the BM proteoglycan perlecan, an interaction between the basic N-terminal, Pro and Arg-rich domain of PRELP and the anionic heparin sulfate (HS) chains of perlecan (Bengtsson et al., 2000). The PRELP/HS interaction is postulated to link PRELP to cell surface HS-proteoglycans (Bengtsson et al., 2000). The core protein of PRELP interacts with collagen fibrils and may serve to link cells to BMs in the adjacent ECM (Bengtsson et al., 2002). Overexpression of PRELP in mice results in structural change in the skin, with a decrease in collagen fiber bundle content and size in the dermis (Grover et al., 2007).

Methods of Diagnosis and Assessment

As set out in the Examples below, the expression patterns of OMD and PRELP in many types of cancers including bladder and kidney cancers, was examined using quantitative RT-PCR, microarray, and immunihistochemistry of cancer tissues.

126 bladder cancer and 31 normal control samples were microdisected using laser capture microscope and expression of OMD and PRELP were analyzed by quantitative RT-PCR using primers indicated in Table 1. The conditions were confirmed as shown in FIG. 2.

The expression levels of both OMD and PRELP were found to be significantly lower in tumors compared with normal tissues (P<0.0001 in each case; FIG. 3A-D and Table 2). Since OMD and PRELP expression is suppressed in early cancer cells from very early stages, analysis based on the tumor stage did not reveal a significant difference between early stages (pTa/pT1) and pT2 stage for either OMD or PRELP. However, the expression levels of both OMD and PRELP were significantly lower in advanced stages pT3/pT4, compared to pT2, though numbers were small in the T3/T4 group. We found a significant difference of OMD expression levels between tumor grades G1 and G2, but no significant difference between tumor grades G2 and G3. In the case of PRELP expression, we could not find any significant features based on grade progression except for a slight difference between tumor grades G1 and G2. Both OMD and PRELP expression levels were lower in primary tumors which were known to have metastasized. As shown in Table 2, no significant differences were observed when categorizing by gender or recurrence status. Although we also analyzed the quantitative RT-PCR results with respect to age, tumor size, smoking history and invasion status, we could not find any significant difference (data not shown).

These results indicate that the expression levels of OMD and PRELP are drastically down-regulated from the very early stages of bladder carcinogenesis, and that the expression levels of these genes remain low in the terminal stages of carcinogenesis, demonstrating that these genes are ideal for all stages and grades of cancers. Moreover, the expression levels have a significant correlation with the stage of tumourigenesis, demonstrating that these genes are suitable for determination of stages of bladder cancer.

Next, we performed quantitative gene expression analysis of OMD and PRELP in 78 renal cell carcinoma and 15 normal control samples (FIG. 4; Table 3). Expression levels in tumor tissues were dramatically lower than those in normal tissues for both OMD (P<0.0001; FIG. 4B) and PRELP (P<0.0001; FIG. 4D). Analysis of the results based on the tumor stage, showed that both OMD and PRELP expression levels in pT3 and pT4 tumor tissues were significantly lower than in pT1 and pT2. However, we could not find significant differences for tumor grade, metastasis state, survival duration or histological cell type (Table 3). In addition, gender and age of samples could not differentiate the expression levels of OMD and PRELP (data not shown). These data show that the OMD and PRELP expression levels are significantly down-regulated from the initial stages of renal carcinogenesis. Moreover, the expression level of early stage cancer is significantly higher than advanced stages, indicating that OMD and PRELP can work as indicators of cancer stages as observed in bladder cancer.

Finally, our statistical detailed analysis revealed that based on expression analysis of OMD and PRELP we can predict whether the tissue is cancer or not with almost 100% accuracy. As shown in FIGS. 3 and 4, we first set a cutoff to distinguish tumor from normal. To derive a cutoff value, we calculated the interquartile range (IQR) by subtracting the first quartile (x_{0.25}) from the third quartile (x_{0.75}) in each data. We considered any data observation which lies more than 1.5*IQR lower than the first quartile or 1.5*IQR higher than the third quartile as an outlier, and derived a cutoff value as follows: Cutoff=[(smallest non-outlier observation in normal tissues)+(largest non-outlier observation in tumor tissues)]/2

Diagnostic values of OMD and PRELP are summarized in Table 4. In the case of the bladder, the expression levels of OMD and PRELP in most normal tissues were above the cutoff value (OMD, 26 of 31 [specificity 83.9%]; PRELP, 28 of 31 [specificity 90.3%]), while expression in most tumor tissues was below the cutoff (OMD, 112 of 126 [sensitivity 88.9%]; PRELP, 114 of 126 [sensitivity 90.5%]; Table 4). In addition, levels of OMD and PRELP in the early stage of almost all tumor tissues were also below the cutoff value (OMD, 80 of 90 [sensitivity 88.9%]; PRELP, 82 of 90 [sensitivity 91.2%]; Table 4). These results indicate that the expression levels of these genes are indeed a useful indicator for the presence of bladder cancer. Moreover, we combined the data of both OMD and PRELP (Table 4). No normal tissue samples were found in the category with both genes below the cutoff [specificity 100%]. Importantly, At least one, PRELP or OMD, in 120 of 126 tumor samples were below the cut off [sensitivity 95.2%]. These data show that we could clearly distinguish tumor from normal samples with combination of both PRELP and OMD data.

For kidney, the expression levels of OMD and PRELP in many normal tissues were above the cutoff (OMD, 13 of 15 [specificity 86.7%]; PRELP, 12 of 15 [specificity 80.0%]), while expression levels in many tumor tissues were below the cutoff (OMD, 64 of 78 [sensitivity 82.1%]; PRELP, 65 of 78 [sensitivity 82.5%]). Expression of both genes in the early stage of most tumor tissues was also below the cutoff (OMD, 22 of 25 [sensitivity 88.0%]; PRELP, 22 of 25 [sensitivity 88.0%]). Combining the data for OMD and PRELP resulted in no normal tissue sample being included in the category of both below the cutoff [specificity 100%]. On the other hand, a large number of tumor cases were in this category of at least one below the cutoff (74 of 79 [sensitivity 93.6%]). In addition, a significant number of early stage tumor cases are also in this category (23 of 25 [sensitivity 92%]). These results show that we could also distinguish the renal tumor samples from normal just as well as for bladder cancer, and around 84% of tumor samples could be detected from an early stage.

Our expression analysis of OMD and PRELP in cancer tissues demonstrated the significant value of OMD- and PRELP-based cancer diagnosis. To get more supporting evidence, the expression levels of OMD and PRELP among cancer cell lines were determined and compared with normal tissues and tumor tissues. We conducted quantitative gene expression analysis of OMD and PRELP in nine normal tissue types, ten bladder cancer cell lines and bladder tumor tissues (FIG. 5). OMD expression levels in normal tissues are high in the lung, fetal eye and bladder, moderate in the stomach, colon, heart, brain and kidney and low in the liver. Levels are also quite low in bladder tumor tissues as examined above. In addition, the OMD expression levels are significantly lower in most bladder caner cell lines compared with normal bladder tissue (FIG. 5A and 5B). Interestingly, the expression level in RT-4 cells is significantly higher than other cell lines: this cell line is a well-differentiated bladder cell line, and this result is consistent with our data.

FIG. 5C shows PRELP expression in several normal tissues and bladder tumor tissues. Levels are quite high in the lung and bladder, and moderate in the stomach, colon, fetal eye and kidney and low in heart, brain and liver. Levels are extremely low in bladder tumor tissues and significantly low in all bladder caner cell lines, which have levels are less than or equal to the levels in bladder tumors. These results reveal that OMD and PRELP genes are ubiquitously expressed in normal tissues and the expression levels are significantly higher than in bladder tumor tissues. Furthermore, the expression levels in most bladder cancer cell lines are significantly lower than normal bladder tissues. This data emphasizes the reliability of our findings using clinical samples.

To elucidate the more general role of OMD and PRELP in tumourigenesis, their expression patterns in many types of cancer, including epithelial cancers, including bladder, lung, breast, colon, kidney, gastric, and prostate cancers, was examined (FIGS. 6 and 7). The expressions of OMD and PRELP are very strongly suppressed in a majority of cancer samples of all cancer types compared with control cells from the surrounding epithelium. These cancers include lung, breast, stomach, colon, rectum, prostate, utrine cervix, endometrium, ovary, thyroid grand, esophagus, small intestine, and adrenal gland cancers.

To explore further the diagnostic value of OMD and PRELP, immunohistochemistry of cancer and normal tissues was performed by using the antibody specific for PRELP (FIG. 8). The antibody stained normal tissues and the normal part of transitional cancer tissue. However, the cancer part of cancer tissues/sample was not stained at all, consistent with the finding in RT-PCR analysis and the microarray analysis discussed above.

Accordingly, a first aspect of the present invention provides a method for the identification of cancer cells, which method comprises determining the expression of the target protein of the invention in a sample of tissue from a first individual and comparing the pattern of expression observed with the pattern of expression of the same protein in a second clinically normal tissue sample from the same individual or a second healthy individual, with the presence of tumour cells in the sample from the first individual indicated by a difference in the expression patterns observed.

More specifically, the invention provides a diagnostic assay for characterising tumours and neoplastic cells, particularly human neoplastic cells, by the differential expression of the target protein whereby the neoplastic phenotype is associated with, identified by and can be diagnosed on the basis thereof. This diagnostic assay comprises detecting, qualitatively or preferably quantitatively, the expression level of the target protein and making a diagnosis of cancer on the basis of this expression level.

In this context, “determining the expression” means qualitative and/or quantitative determinations, of the presence of the target protein of the invention including measuring an amount of biological activity of the target protein in terms of units of activity or units activity per unit time, and so forth.

As used herein, the term “expression” generally refers to the cellular processes by which a polypeptide is produced from RNA.

In a preferred embodiment of the present invention, this method may be applied to diagnosis of urological cancers such as bladder or kidney cancer.

Unless context demands otherwise, species variants are also encompassed by this invention where the patient is a non-human mammal, as are allelic or other variants of the human OMD and PRELP, and any reference to these proteins will be understood to embrace variants sharing the same activity (e.g. fragments, alleles, homologues, orthologues of other organisms, mutated human genes, mutated orthologues of other organisms, tagged proteins, other modified genes with a similar biological activity or other naturally occurring variants).

The following (SEQ ID NO:1) is the current published amino acid sequence of human OMD:

MGFLSPIYVIFFFFGVKVHCQYETYQWDEDYDQEPDDDYQTGFPFRQNVDYGVPFHQY
TLGCVSECFCPTNFPSSMYCDNRKLKTIPNIPMHIQQLYLQFNEIEAVTANSFINATHLKEI
NLSHNKIKSQKIDYGVFAKLPNLLQLHLEHNNLEEFPFPLPKSLERLLLGYNEISKLQTNA
MDGLVNLTMLDLCYNYLHDSLLKDKIFAKMEKLMQLNLCSNRLESMPPGLPSSLMYLSL
ENNSISSIPEKYFDKLPKLHTLRMSHNKLQDIPYNI
FNLPNIVELSVGHNKLKQAFYIPRNLEHLYLQNNEIEKMNLTVMCPSIDPLHYHHLTYIRV
DQNKLKEPISSYIFFCFPHIHTIYYGEQRSTNGQTIQLKTQVFRRFPDDDDESEDHDDPD
NAHESPEQEGAEGHFDLHYYENQE

SEQ ID NO:2 is the current published amino acid sequence of human PRELP:

MRSPLCWLLPLLILASVAQGQPTRRPRPGTGPGRRPRPRPRPTPSFPQPDEPAEPTDL
PPPLPPGPPSIFPDCPRECYCPPDFPSALYCDSRNLRKVPVIPPRIHYLYLQNNFITELPV
ESFQNATGLRWINLDNNRIRKIDQRVLEKLPGLVFLYMEKNQLEEVPSALPRNLEQLRLS
QNHISRIPPGVFSKLENLLLLDLQHNRLSDGVFKPDTFHGLKNLMQLNLAHNILRKMPPR
VPTAIHQLYLDSNKIETIPNGYFKSFPNLAFIRLNYN
KLTDRGLPKNSFNISNLLVLHLSHNRISSVPAINNRLEHLYLNNNSIEKINGTQICPNDLVA
FHDFSSDLENVPHLRYLRLDGNYLKPPIPLDLMMCFRLLQSVVI

Thus included within the definition of the target protein of the invention are amino acid variants of the naturally occurring sequence as provided in any of SEQ ID NOs:1-2. Preferably, variant sequences are at least 75% homologous to the wild-type sequence, more preferably at least 80% homologous, even more preferably at least 85% homologous, yet more preferably at least 90% homologous or most preferably at least 95% homologous to at least a portion of the reference sequence supplied (SEQ ID NOs:1-2). In some embodiments the homology will be as high as 94 to 96 or 98%. Homology in this context means sequence similarity or identity, with identity being preferred. To determine whether a candidate peptide region has the requisite percentage similarity or identity to a reference polypeptide or peptide oligomer, the candidate amino acid sequence and the reference amino acid sequence are first aligned using a standard computer programme such as are commercially available and widely used by those skilled in the art. In a preferred embodiment the NCBI BLAST method is used (http://www.ncbi.nlm.nih.gov/BLAST/). Once the two sequences have been aligned, a percent similarity score may be calculated. In all instances, variants of the naturally-occurring sequence, as detailed in SEQ ID NO:1-2 herein, must be confirmed for their function as marker proteins. Specifically, their presence or absence in a particular form or in a particular biological compartment must be indicative of the presence or absence of cancer in an individual. This routine experimentation can be carried out by using standard methods known in the art in the light of the disclosure herein.

In one aspect of the present invention, the target protein can be detected using a binding moiety capable of specifically binding the marker protein. By way of example, the binding moiety may comprise a member of a ligand-receptor pair, i.e. a pair of molecules capable of having a specific binding interaction. The binding moiety may comprise, for example, a member of a specific binding pair, such as antibody-antigen, enzyme-substrate, nucleic acid-nucleic acid, protein-nucleic acid, protein-protein, or other specific binding pair known in the art. Binding proteins may be designed which have enhanced affinity for the target protein of the invention. Optionally, the binding moiety may be linked with a detectable label, such as an enzymatic, fluorescent, radioactive, phosphorescent, coloured particle label or spin label. The labelled complex may be detected, for example, visually or with the aid of a spectrophotometer or other detector.

A preferred embodiment of the present invention involves the use of a recognition agent, for example an antibody recognising the target protein of the invention, to contact a sample of tissues, cells, blood or body product, or samples derived therefrom, and screening for a positive response. The positive response may for example be indicated by an agglutination reaction or by a visualisable change such as a colour change or fluorescence, e.g. immunostaining, or by a quantitative method such as in use of radio-immunological methods or enzyme-linked antibody methods.

The method therefore typically includes the steps of (a) obtaining from a patient a tissue sample to be tested for the presence of cancer cells; (b) producing a prepared sample in a sample preparation process; (c) contacting the prepared sample with a recognition agent, such as an antibody, that reacts with the target protein of the invention; and (d) detecting binding of the recognition agent to the target protein, if present, in the prepared sample. The human tissue sample will generally be from the bladder or kidney.

The sample may further comprise sections cut from patient tissues or it may contain whole cells or it may be, for example, a body fluid sample selected from the group consisting of: blood; serum; plasma; fecal matter; urine; vaginal secretion; breast exudate; spinal fluid; saliva; ascitic fluid; peritoneal fluid; sputum; and bladder or kidney exudate, or an effusion, where the sample may contain cells, or may contain shed antigen. A preferred sample preparation process includes tissue fixation and production of a thin section. The thin section can then be subjected to immunohistochemical analysis to detect binding of the recognition agent to the target protein. Preferably, the immunohistochemical analysis includes a conjugated enzyme labelling technique. A preferred thin section preparation method includes formalin fixation and wax embedding. Alternative sample preparation processes include tissue homogenisation. When sample preparation includes tissue homogenisation, a preferred method for detecting binding of the antibody to the target protein is Western blot analysis.

Alternatively, an immunoassay can be used to detect binding of the antibody to the target protein. Examples of immunoassays are antibody capture assays, two-antibody sandwich assays, and antigen capture assays. In a sandwich immunoassay, two antibodies capable of binding the marker protein generally are used, e.g. one immobilised onto a solid support, and one free in solution and labelled with a detectable chemical compound. Examples of chemical labels that may be used for the second antibody include radioisotopes, fluorescent compounds, spin labels, coloured particles such as colloidal gold and coloured latex, and enzymes or other molecules that generate coloured or electrochemically active products when exposed to a reactant or enzyme substrate. When a sample containing the marker protein is placed in this system, the marker protein binds to both the immobilised antibody and the labelled antibody, to form a “sandwich” immune complex on the support's surface. The complexed protein is detected by washing away non-bound sample components and excess labelled antibody, and measuring the amount of labelled antibody complexed to protein on the support's surface. Alternatively, the antibody free in solution, which can be labelled with a chemical moiety, for example, a hapten, may be detected by a third antibody labelled with a detectable moiety which binds the free antibody or, for example, the hapten coupled thereto. Preferably, the immunoassay is a solid support-based immunoassay. Alternatively, the immunoassay may be one of the immunoprecipitation techniques known in the art, such as, for example, a nephelometric immunoassay or a turbidimetric immunoassay. When Western blot analysis or an immunoassay is used, preferably it includes a conjugated enzyme labelling technique.

Although the recognition agent will conveniently be an antibody, other recognition agents are known or may become available, and can be used in the present invention. For example, antigen binding domain fragments of antibodies, such as Fab fragments, can be used. Also, so-called RNA aptamers may be used. Therefore, unless the context specifically indicates otherwise, the term “antibody” as used herein is intended to include other recognition agents. Where antibodies are used, they may be polyclonal or monoclonal. Optionally, the antibody can be produced by a method such that it recognizes a preselected epitope from the target protein of the invention.

The isolated target protein of the invention may be used for the development of diagnostic and other tissue evaluation kits and assays to monitor the level of the proteins in a tissue or fluid sample. For example, the kit may include antibodies or other specific binding moieties which bind specifically to the target protein which permit the presence and/or concentration of the bladder or kidney cancer-associated proteins to be detected and/or quantified in a tissue or fluid sample. Accordingly, the invention further provides for the production of suitable kits for detecting the target protein, which may for example include a receptacle or other means for receiving a sample to be evaluated, and a means for detecting the presence and/or quantity in the sample of the target protein of the invention and optionally instructions for performing such an assay.

There are several ways to detect the level of OMD and PRELP based on nucleic acid encoding therefor. These include detection of mRNA level, detection of protein level, detection of transcriptional activity, detection of translation activity. The methods to detect mRNA level include quantitative RT-PCR and microarray analysis. Some of these will now be described.

In a particular embodiment, the level of marker mRNA can be determined both by in situ and by in vitro formats in a biological sample using methods known in the art. For in vitro methods, any RNA isolation technique that does not select against the isolation of mRNA can be utilised for the purification of RNA (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridisation or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridise to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridise under stringent conditions to a mRNA encoding a marker of the present invention.

For example the methods may employ a probe of around 30 nucleotides or longer. The stringent conditions may comprise washing in 0.1% SDS/0.1×SSC at 68° C.

Hybridisation of an mRNA with the probe indicates that the marker in question is being expressed. In most preferred embodiments detection and/or quantification of the metastasis-specific biological markers is performed by using suitable DNA microarrays. In such a marker detection/quantification format, the mRNA is immobilised on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

An alternative method for determining the level of mRNA marker in a sample involves the process of nucleic acid amplification, e.g., by RT-PCR (as described below), ligase chain reaction (Barany, 1991 , Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1 173-1 177), Q-Beta Replicase (Lizardi et al., 1988, Bio/Technology 6:1 197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the sample prior to detection. In such methods, a cell or tissue sample is prepared/processed using known histological methods. The sample is then immobilised on a support, typically a glass slide, and then contacted with a probe that can hybridise to mRNA that encodes the marker.

As an alternative to making determinations based on the absolute expression level of the marker, determinations may be based on the normalised expression level of the marker. Expression levels are normalised by correcting the absolute expression level of a marker by comparing its expression to the expression of a gene that is not a marker, e.g., a housekeeping gene that is constitutively expressed. Suitable genes for normalisation include housekeeping genes such as the actin gene. This normalisation allows the comparison of the expression level of one or more tissue-specific biological marker of interest in one sample.

Alternatively, the expression level can be provided as a relative expression level. To determine a relative expression level of a marker, the level of expression of the marker is determined for 4, 5, 10 or more samples of normal versus cancer cell isolates, prior to the determination of the expression level for the sample in question. The median expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level which can itself be categorised e.g. <50%, <33%, <20% and so on.

Thus in one aspect the invention may comprise the steps of obtaining a test sample comprising nucleic acid molecules present in a sample of the individual then determining the amount of mRNA encoding the target protein in the test sample and optionally comparing the amount of mRNA in the test sample to a predetermined value.

More preferably the step of determining the amount of mRNA in the test sample entails a specific amplification of the mRNA and then quantitation of the amplified produce e.g. via RT-PCR analysis as described in the Examples below.

Transcription levels are regulated by epigenetic modification and transcription factors. Measurement of status of specific epigenetic and/or transcription factors can detect transcriptional activity. Put another way, it is known in the art that decreased levels of expression and transcription are often the result of promoter hypermethylation. Therefore in one embodiment of the invention it may be desirable to determine whether the OMD or PRELP gene promoters are hypermethylated. Promoter methylation can be detected by known techniques including restriction endonuclease treatment and Southern blot analysis. Techniques include those published in U.S. Pat. No. 5,552,277 or more recent techniques (see e.g. “DNA methylation protocols” (2002) By Ken I. Mills, Bernie H. Ramsahoye; “DNA methylation: approaches, methods, and applications” (2005) By Manel Esteller). Therefore, in a method of the invention, when the cellular component detected is DNA, restriction endonuclease analysis is preferable to detect hypermethylation of the promoter. Any restriction endonuclease that includes CG as part of its recognition site and that is inhibited when the C is methylated, can be utilized. Preferably, the methylation sensitive restriction endonuclease is BssHII, MspI, or HpaII, used alone or in combination. Other methylation sensitive restriction endonucleases will be known to those of skill in the art.

In these embodiments of the invention, the preceding claims wherein the pattern or level of expression of the proteins are thus inferred by detecting methylation of the promoter region of the gene encoding the or each target protein. Generally hypermethylation (compared to a reference or control, as described herein) is correlated with reduced expression of the protein in question. Optionally this is assessed using a reagent which detects methylation of the promoter region, which is optionally a restriction endonuclease e.g. a methylation sensitive endonuclease such as MspI, HpaII and BssHII.

Translation is also regulated by multiple mechanisms such as microRNA action. All such methodologies for detecting translational suppression of OMD and PRELP proteins are also involved in this invention.

In a further aspect of the present invention is provided herein a method of evaluating the effect of a candidate therapeutic drug for the treatment of cancer, said method comprising administering said drug to a patient, removing a cell sample from said patient; and determining the expression profile of (e.g. quantifying) the target protein of the invention in said cell sample. This method may further comprise comparing said expression profile to an expression profile of a healthy individual.

In a preferred embodiment, said patient is receiving treatment for an epithelial cancer e.g. a urological cancer e.g. bladder or kidney cancer, and said cell sample is derived from epithelial tissues e.g. bladder or kidney. In a further preferred embodiment the present invention further provides a method for determine the efficacy of a therapeutic regime at one or more time-points, said method comprising determining a baseline value for the expression of the protein being tested in a given individual within a given tissue such as a tumour, administering a given therapeutic drug, and then redetermining expression levels of the protein within that given tissue at one or more instances thereafter, observing changes in protein levels as an indication of the efficacy of the therapeutic regime.

Thus, for example, and without limitation, the present invention embraces:
Detection of mRNA of OMD and\or PRELP of samples from actual or suspected cancer patients (e.g. an epithelial cancer e.g. a urological cancer e.g. bladder or kidney cancer). This may be achieved by any method known in the art e.g. microarray and RT-PCR.
Detection of OMD and\or PRELP protein in samples from actual or suspected cancer patients (e.g. an epithelial cancer e.g. a urological cancer e.g. bladder or kidney cancer). This may be achieved by any method known in the art e.g. specific antibodies and OMD, PRELP binding proteins.
Detection of OMD and\or PRELP activity in samples from actual or suspected cancer patients (e.g. an epithelial cancer e.g. a urological cancer e.g. bladder or kidney cancer). This may be achieved by any method known in the art.
Detection of the degree of suppression of OMD and\or PRELP transcription and\or mRNA translation activity in samples from actual or suspected cancer patients (e.g. an epithelial cancer e.g. a urological cancer e.g. bladder or kidney cancer). This may be achieved by any method known in the art e.g. detection of specific transcriptional downregulation and epigenetic modification. Epigenetic modification can be detected by directly examination of cancer samples and by indirect examination of body fluids such as blood and urine samples. This also includes specific translation regulatory mechanisms such as detection of miRNA activity.
Determination of cancer staging using the above detection methods
Determination of patient prognosis using the above detection methods
Measurement of efficacy of cancer treatment using the above detection methods.

Screening Methods and Therapeutic Strategies

Also disclosed herein are novel methods for treatment of cancer based on activation of OMD and/or PRELP. Our expression analysis described above suggested that downregulation of OMD and PRELP may have advantages for development of cancer. If so, it can be inferred that the activation of OMD or/and PRELP would inhibit tumourigenesis and provide novel treatment of cancer. To demonstrate this, we examined the effect of overexpression of OMD or PRELP in cancer cells and xenograftic mouse models on cancer-related properties. To this end, OMD or PRELP was stably over-expressed in the bladder cancer cell line EJ28. OMD-transfected stable cell lines showed enhanced cell cycle arrest at G1 phase. OMD and PRELP both inhibit proliferation, as measured in a cell counting assay. Furthermore, stable overexpression of OMD or PRELP results in increased cell death by apoptosis. FIG. 9 shows the abnormal morphology induced by OMD overexpression.

Also, activation of OMD or PRELP in cancer cells increased sensitivity to the DNA damaging reagent, Mitomycin C, indicating that combination of activation of OMD or PRELP with cancer drugs can provide better treatment of cancer (FIG. 10). Interestingly, this chemosensitization was unique to cancer cells. OMD overexpression actually protected normal cells from Mitomycin-C mediated apoptosis whilst PRELP had no effect on their sensitivity (FIG. 10). This suggests that treatment with OMD and/or PRELP, in combination with Mitomycin C treatment, would enhance killing of cancer cells, but protect normal cells.

OMD and PRELP also affect the anchorage-independent growth of cancer cells. Anchorage-independence is a hallmark of cancer cells. Normal epithelial cells require a substrate on which to grow, but carcinoma cells can proliferate in the absence of a substrate, and thus form tumours. Measuring the ability of cancer cells to grow in soft agar is the gold standard approach for measuring anchorage-independence and tumour forming ability in vitro. Strikingly, OMD overexpression absolutely abolishes anchorage-independence of EJ28 cells, suggesting that OMD could dramatically inhibit tumour formation. PRELP also inhibits anchorage-independent growth of EJ28, and reduces colony-forming ability in soft agar to a third of that observed in control cells (FIG. 14).

To evaluate in vivo effect of OMD or PRELP activation in cancer cells, xenograft experiments were performed using nude mice. EJ28 bladder cancer cells expressing OMD or EJ28 control cells were injected into the mice and cancer development was monitored for three weeks. The mice injected with EJ28 control cells developed cancer, while those injected the cells expressing OMD did not form any cancer (FIG. 15). This observation confirms the utility of OMD/PRELP based cancer treatment.

To determine the mechanism of apoptosis induced by OMD or PRELP, we examined downstream signalling pathways. To this end, stable cell lines overexpressing OMD or PRELP and non-stable cell lines having suppression of OMD or PRELP have been constructed and influenced signalling pathways analysed.

In order to overexpress the genes, we used non-transformed cells of 293 cells because overexpression of these genes in transformed cancer cells resulted in significant increase of apoptosis. Also, the T-Rex-293 system was used for the construction because this system is suitable for expression at relatively physiological level without causing adverse effect based on their insertion site. FIG. 12 shows that OMD-1 cells and PRELP-1 cells have expression of the protein and their expression levels are relevant to natural expression level.

In order to knock down expression of genes, 5637 bladder cancer cells were transfected with siRNA constructs of siOMD, siPRELP, siEGFP, or siFFLuc. As mentioned, expression in a majority of bladder cancer cell lines is strongly suppressed. The 5637 cells have relatively high expression compared with the majority of bladder cancer cell lines. After suppression, expression of OMD and PRELP were confirmed by quantitative RT-PCR. FIG. 13 shows that expression of PRELP was strongly suppressed in siPRELP, but the control constructs of siEGFP or siFFLuc did not suppress PRELP levels.

RNAs were isolated from these cells and then expression profiling of mRNA were determined using Affymetrix's Genechip system. From the data, statistically significantly up-regulated or down-regulated genes are identified through comparison with controls. To validate the experiments, we have confirmed expression level of some genes identified by microarray using quantitative RT-PCR (FIG. 13).

Then, the genes up-regulated by OMD over-expression and suppressed by OMD deletion and the genes down-regulated by OMD over-expression and up-regulated by OMD deletion were determined (Table 5). Also, the genes up-regulated by PRELP over-expression and suppressed by PRELP deletion and the genes down-regulated by PRELP over-expression and up-regulated by PRELP deletion were determined (Table 6). Interestingly, the lists include many tumour suppressor genes and oncogenes and also there is significant overlap.

To determine influenced signaling pathways, the microarray data were subjected to statistical analysis by the KEGG pathway analysis. Our statistic analysis revealed that both OMD and PRELP most strongly influence the p53 pathway. Also, OMD and PRELP regulate the tight junction and the apoptosis pathways. In addition, OMD regulates the adherens junction, the Wnt, the apoptosis pathways (Table 7). Suppression of OMD or PRELP has significant impact on tumourigenesis. Also, OMD and PRELP are functionally largely redundant in tumourigenesis. Furthermore, we have examined the effect of OMD or PRELP on the signalling pathway activities using biochemistry based assays. OMD or PRELP regulate multiple tumour related signalling pathways such as Wnt, TGF-b, NFkB, myc and ras pathways, which results in regulation of apoptosis and tight junction. All observations indicate that activation of OMD, and/or PRELP is an ideal method to kill cancer cells through activation of tumour suppressing activities such as the p53 pathway, the apoptotic pathway, and the tight junction pathway.

Thus a further embodiment of the present invention is the development of therapies for treatment of conditions which are characterized by under-expression of the target protein of the invention via immunotherapeutic approaches.

Such methods may comprise administering or activating OMD and/or PRELP in the cell, or mimicing the activity thereof. For example proteins or polypeptides may be administered in an amount sufficient to give therapeutic benefit. By way of example, which is specifically not intended to limit the scope of the invention, these may be administered as naked peptides, as peptides conjugated or encapsulated in one or more additional molecules (e.g. liposomes) such that a pharmacological parameter (e.g. tissue permeability, resistance to endogenous proteolysis, circulating half-life etc) is improved, or in a suitable expression vector which causes the expression of the sequences at an appropriate site within the body

Because down-regulation of expression of the target protein of the invention is associated with tumour cells, it is likely that these proteins in some way contribute to the process of tumourigenesis. Consequently, the present invention provides for the increase of the expression level of the target protein in tumour cells.

Thus one preferred method comprises the step of administering to a patient diagnosed as having cancer, such as bladder or kidney cancer, a therapeutically-effective amount of a compound which increases in vivo the expression of the target protein.

In a preferred embodiment, the compound is a polynucleotide, for example encoding OMD and/or PRELP. By way of further example, constructs of the present invention capable of increasing expression of the target protein can be administered to the subject either as a naked polynucleotide or formulated with a carrier, such as a liposome, to facilitate incorporation into a cell. Such constructs can also be incorporated into appropriate vaccines, such as in viral vectors (e.g. vaccinia), bacterial constructs, such as variants of the well known BCG vaccine, and so forth.

Thus one DNA based therapeutic approach provided by the present invention is the use of a vector which comprises one or more nucleotide sequences, preferably a plurality of these, each of which encodes OMD and/or PRELP.

Alternatively increase in expression levels could be achieved by up-regulation of the corresponding gene promoter.

Screening Methods

A further aspect of the present invention provides novel methods for screening for compositions that modulate the expression or biological activity of the target protein of the invention. As used herein, the term “biological activity” means any observable effect resulting from interaction between the target protein and a ligand or binding partner. Representative, but non-limiting, examples of biological activity in the context of the present invention include regulation of the genes shown in Table 5 or interaction with a binding partner.

The term “biological activity” also encompasses both the inhibition and the induction of the expression of the target protein of the invention. Further, the term “biological activity” encompasses any and all effects resulting from the binding of a ligand or other in vivo binding partner by a polypeptide derivative of the protein of the invention. In one embodiment, a method of screening drug candidates comprises providing a cell that expresses the target protein of the invention, adding a candidate therapeutic compound to said cell and determining the effect of said compound on the expression or biological activity of said protein. In a further embodiment, the method of screening candidate therapeutic compounds includes comparing the level of expression or biological activity of the protein in the absence of said candidate therapeutic compound to the level of expression or biological activity in the presence of said candidate therapeutic compound.

Where said candidate therapeutic compound is present its concentration may be varied, and said comparison of expression level or biological activity may occur after addition or removal of the candidate therapeutic compound. The expression level or biological activity of said target protein may show an increase or decrease in response to treatment with the candidate therapeutic compound.

Candidate therapeutic molecules of the present invention may include, by way of example, peptides produced by expression of an appropriate nucleic acid sequence in a host cell or using synthetic organic chemistries, or non-peptide small molecules produced using conventional synthetic organic chemistries well known in the art. Screening assays may be automated in order to facilitate the screening of a large number of small molecules at the same time.

As used herein, the terms “candidate therapeutic compound” refers to a substance that is believed to interact with the target protein of the invention (or a fragment thereof), and which can be subsequently evaluated for such an interaction. Representative candidate therapeutic compounds include “xenobiotics”, such as drugs and other therapeutic agents, natural products and extracts, carcinogens and environmental pollutants, as well as “endobiotics” such as steroids, fatty acids and prostaglandins. Other examples of candidate compounds that can be investigated using the methods of the present invention include, but are not restricted to, agonists and antagonists of the target protein of the invention, toxins and venoms, viral epitopes, hormones (e. g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

In one preferred embodiment the present invention provides a method of drug screening utilising eukaryotic or prokaryotic host cells stably transformed with recombinant polynucleotides expressing the target protein of the invention or a fragment thereof, preferably in competitive binding assays. Such cells, either in viable or fixed form, can be used for standard binding assays. For example, the assay may measure the formation of complexes between a target protein and the agent being tested, or examine the degree to which the formation of a complex between the target protein or fragment thereof and a known ligand or binding partner is interfered with by the agent being tested. Thus, the present invention provides methods of screening for drugs comprising contacting such an agent with the target protein of the invention or a fragment thereof or a variant thereof found in a tumour cell and assaying (i) for the presence of a complex between the agent and the target protein, fragment or variant thereof, or (ii) for the presence of a complex between the target protein, fragment or variant and a ligand or binding partner. In such competitive binding assays the target protein or fragment or variant is typically labelled. Free target protein, fragment or variant thereof is separated from that present in a protein: protein complex and the amount of free (i.e. uncomplexed) label is a measure of the binding of the agent being tested to the target protein or its interference with binding of the target protein to a ligand or binding partner, respectively.

Alternatively, an assay of the invention may measure the influence of the agent being tested on a biological activity of the target protein. Thus, the present invention provides methods of screening for drugs comprising contacting such an agent with the target protein of the invention or a fragment thereof or a variant thereof found in a tumour cell and assaying for the influence of such an agent on a biological activity of the target protein, by methods well known in the art. In such activity assays the biological activity of the target protein, fragment or variant thereof is typically monitored by provision of a reporter system. For example, this may involve provision of a natural or synthetic substrate that generates a detectable signal in proportion to the degree to which it is acted upon by the biological activity of the target molecule.

It is contemplated that, once candidate therapeutic compounds have been elucidated, rational drug design methodologies well known in the art may be employed to enhance their efficacy. The goal of rational drug design is to produce structural analogues of biologically active polypeptides of interest or of small molecules with which they interact (e. g. agonists, antagonists, inhibitors) in order to fashion drugs which are, for example, more active or stable forms of the polypeptide, or which, for example, enhance or interfere with the function of a polypeptide in vivo. In one approach, one first determines the three-dimensional structure of a protein of interest, such as the target protein of the invention or, for example, of the target protein in complex with a ligand, by x-ray crystallography, by computer modelling or most typically, by a combination of approaches. For example, the skilled artisan may use a variety of computer programmes which assist in the development of quantitative structure activity relationships (QSAR) that act as a guide in the design of novel, improved candidate therapeutic molecules. Less often, useful information regarding the structure of a polypeptide may be gained by modelling based on the structure of homologous proteins. In addition, peptides can be analysed by alanine scanning (Wells, Methods Enzymol. 202: 390-411, 1991), in which each amino acid residue of the peptide is sequentially replaced by an alanine residue, and its effect on the peptide's activity is determined in order to determine the important regions of the peptide. It is also possible to design drugs based on a pharmacophore derived from the crystal structure of a target-specific antibody selected by a functional assay. It is further possible to avoid the use of protein crystallography by generating anti-idiotypic antibodies to such a functional, target-specific antibody, which have the same three-dimensional conformation as the original target protein. These anti-idiotypic antibodies can subsequently be used to identify and isolate peptides from libraries, which themselves act as pharmacophores for further use in rational drug design.

For use as a medicament in vivo, candidate therapeutic compounds so identified may be combined with a suitable pharmaceutically acceptable carrier, such as physiological saline or one of the many other useful carriers well characterized in the medical art. Such pharmaceutical compositions may be provided directly to malignant cells, for example, by direct injection, or may be provided systemically, provided the formulation chosen permits delivery of the therapeutically effective molecule to tumour cells containing the target protein of the invention. Suitable dose ranges and cell toxicity levels may be assessed using standard dose ranging methodology. Dosages administered may vary depending, for example, on the nature of the malignancy, the age, weight and health of the individual, as well as other factors.

Thus without limitation, the following cancer therapeutic approaches are included in this invention.
Methods to increasing the activity of OMD and/or PRELP.
Introduction of nucleic acids (e.g. DNA or mRNA) encoding OMD and/or PRELP using any conventional methods e.g. introduction of expression plasmid, expression cosmid, expression bac, and expression virus.
Introduction of protein(s) of OMD and/or PRELP.
Introduction of molecules that have an activity similar to OMD and/or PRELP or augment that activity.
Transcriptional or activation of the endogenous gene of, or translational activation of endogenous mRNA of, OMD and\or PRELP.
Stabilisation of the endogenous protein of OMD and\or PRELP
Stabilization of the endogenous mRNA of OMD and\or PRELP
Activation by post-translational modification specific for OMD and\or PRELP.
Increase of gene copy number of OMD and\or PRELP

Use of OMD and/or PRELP in any of these methods.

Test Animals and Cells

A further aspect of the present invention provides for cells and animals which express the target protein of the invention (or contain “knock outs” of the target protein) and can be used as model systems to study and test for substances which have potential as therapeutic agents for the cancers discussed herein.

Such cells may be isolated from individuals with mutations, either somatic or germline, in the gene encoding the target protein of the invention, or can be engineered to express, over-express or knockout the target protein or a variant thereof, using methods well known in the art. After a test substance is applied to the cells, any relevant trait of the cells can be assessed, including by way of example growth, viability, tumourigenicity in nude mice, invasiveness of cells, and growth factor dependence, assays for each of which traits are known in the art.

Animals for testing candidate therapeutic agents can be selected after mutagenesis of whole animals or after treatment of germline cells or zygotes. As discussed in more detail below, by way of example, such treatments can include insertion of genes encoding the target protein of the invention in wild-type or variant form, typically from a second animal species, as well as insertion of disrupted homologous genes. Alternatively, the endogenous target protein gene(s) of the animals may be disrupted by insertion or deletion mutation or other genetic alterations using conventional techniques that are well known in the art. After test substances have been administered to the animals, the growth of tumours can be assessed. If the test substance prevents or suppresses the growth of tumours, then the test substance is a candidate therapeutic agent for the treatment of those cancers expressing the target protein of the invention, for example of bladder or kidney cancers. These animal models provide an extremely important testing vehicle for potential therapeutic compounds.

Thus the present invention thus provides a transgenic non-human animal, particularly a rodent, which comprises an inactive copy of the gene encoding a target protein of the present invention.

The invention further provides a method of testing a putative therapeutic of the invention which comprises administering said therapeutic to an animal according to the invention and determining the effect of the therapeutic.

For the purposes of the present invention, it will be understood that reference to an inactive copy of the gene encoding a target protein of the present invention includes any non-wild-type variant of the gene which results in knock out or down regulation of the gene, and optionally in a cancer phenotype e.g. in a test animal. Thus the gene may be deleted in its entirety, or mutated such that the animal produces a truncated protein, for example by introduction of a stop codon and optionally upstream coding sequences into the open reading frame of the gene encoding a target protein of the present invention. Equally, the open reading frame may be intact and the inactive copy of the gene provided by mutations in promoter regions.

Generally, inactivation of the gene may be made by targeted homologous recombination. Techniques for this are known as such in the art. This may be achieved in a variety of ways. A typical strategy is to use targeted homologous recombination to replace, modify or delete the wild-type gene in an embryonic stem (ES) cell. A targeting vector comprising a modified target gene is introduced into ES cells by electroporation, lipofection or microinjection. In a few ES cells, the targeting vector pairs with the cognate chromosomal DNA sequence and transfers the desired mutation carried by the vector into the genome by homologous recombination. Screening or enrichment procedures are used to identify the transfected cells, and a transfected cell is cloned and maintained as a pure population. Next, the altered ES cells are injected into the blastocyst of a preimplantation mouse embryo or alternatively an aggregation chimera is prepared in which the ES cells are placed between two blastocysts which, with the ES cells, merge to form a single chimeric blastocyst. The chimeric blastocyst is surgically transferred into the uterus of a foster mother where the development is allowed to progress to term. The resulting animal will be a chimera of normal and donor cells. Typically the donor cells will be from an animal with a clearly distinguishable phenotype such as skin colour, so that the chimeric progeny is easily identified. The progeny is then bred and its descendants cross-bred, giving rise to heterozygotes and homozygotes for the targeted mutation. The production of transgenic animals is described further by Capecchi, M, R., 1989, Science 244; 1288-1292; Valancius and Smithies, 1991, Mol. Cell. Biol. 11; 1402-1408; and Hasty et al, 1991, Nature 350; 243-246, the disclosures of which are incorporated herein by reference.

Homologous recombination in gene targeting may be used to replace the wild-type gene encoding a target protein of the present invention with a specifically defined mutant form (e.g. truncated or containing one or more substitutions).

The inactive gene may also be one in which its expression may be selectively blocked either permanently or temporarily. Permanent blocking may be achieved by supplying means to delete the gene in response to a signal. An example of such a means is the cre-lox system where phage lox sites are provided at either end of the transgene, or at least between a sufficient portion thereof (e.g. in two exons located either side or one or more introns). Expression of a cre recombinase causes excision and circularisation of the nuclei acid between the two lox sites. Various lines of transgenic animals, particularly mice, are currently available in the art which express cre recombinase in a developmentally or tissue restricted manner, see for example Tsien, Cell, Vol. 87(7): 1317-1326, (1996) and Betz, Current Biology, Vol. 6(10): 1307-1316 (1996). These animals may be crossed with lox transgenic animals of the invention to examine the function of the gene encoding a target protein of the present invention. An alternative mechanism of control is to supply a promoter from a tetracycline resistance gene, tet, to the control regions of the target gene locus such that addition of tetracycline to a cell binds to the promoter and blocks expression of the gene encoding a target protein of the present invention. Alternatively GAL4, VP16 and other transactivators could be used to modulate gene expression including that of a transgene containing the gene encoding a target protein of the present invention. Furthermore, the target gene could also be expressed in ectopic sites, that is in sites where the gene is not normally expressed in time or space.

Transgenic targeting techniques may also be used to delete the gene encoding a target protein of the present invention. Methods of targeted gene deletion are described by Brenner et al, WO94/21787 (Cell Genesys), the disclosure of which is incorporated herein by reference.

In a further embodiment of the invention, there is provided a non-human animal which expresses the gene encoding a target protein of the present invention at a higher than wild-type level. Preferably this means that the gene encoding a target protein of the present invention is expressed at least 120-200% of the level found in wild-type animals of the same species, when cells which express the gene are compared. Also, this gene could be expressed in an ectopic location where the target gene is not normally expressed in time or space. Comparisons may be conveniently done by northern blotting and quantification of the transcript level. The higher level of expression may be due to the presence of one or more, for example two or three, additional copies of the target gene or by modification to the gene encoding a target protein of the present inventions to provide over-expression, for example by introduction of a strong promoter or enhancer in operable linkage with the wild-type gene. The provision of animals with additional copies of genes may be achieved using the techniques described herein for the provision of “knock-out” animals.

Non-human mammalian animals include non-human primates, rodents, rabbits, sheep, cattle, goats, pigs. Rodents include mice, rats, and guinea pigs. Amphibians include frogs. Fish such as zebra fish, may also be used. Transgenic non-human mammals of the invention may be used for experimental purposes in studying cancer, and in the development of therapies designed to alleviate the symptoms or progression of cancer. By “experimental” it is meant permissible for use in animal experimentation or testing purposes under prevailing legislation applicable to the research facility where such experimentation occurs.

Any sub-titles herein are included for convenience only, and are not to be construed as limiting the disclosure in any way.

The invention will now be further described with reference to the following non-limiting Tables, Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

Tables

Table 1. Primer sequences for quantitative RT-PCR. The primers used for quantitative RT-PCR analysis are shown.

Table 2. Statistical analysis of OMD and PRELP expression levels in clinical bladder tissues

Table 3. Statistical analysis of OMD and PRELP expression levels in clinical renal tissues

Table 4. Relationship between OMD and PRELP expression levels and carcinogenesis

Table 5. A list of genes regulated by OMD. The genes that are significantly activated by OMD overexpression and are suppressed by OMD deletion and the genes that are suppressed by OMD overexpression and are activated by OMD suppression are indicated.

Table 6. A list of genes regulated by PRELP. The genes that are significantly activated by PRELP overexpression and are suppressed by PRELP deletion and the genes that are suppressed by PRELP overexpression and are activated by PRELP suppression are indicated.

Table 7. The KEGG pathway analysis of OMD based on the Affymetrix's microarray data. From the genes listed in Tables 5 and 6, influenced signaling pathways were determined using the KEGG pathway analysis programme.

### FIGURES

FIG. 1. Structure of OMD, PRELP, and keratocan

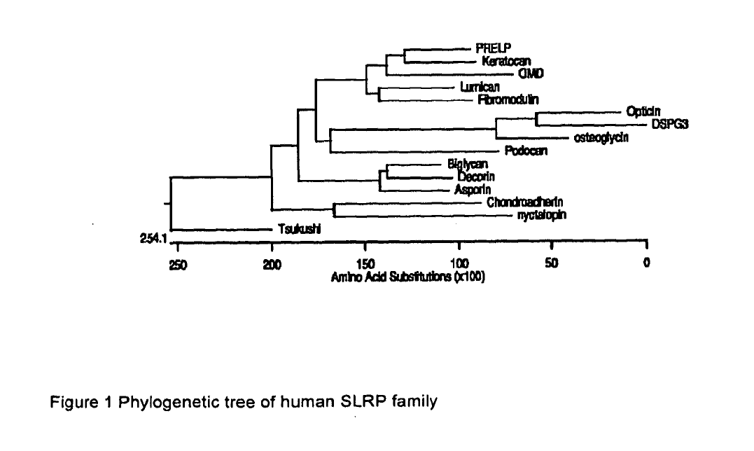

OMD, PRELP, and keratocan form a branch of the SLRP family. They are very homologous but different from other family members.

FIG. 2. The validation of real-time quantitative RT-PCR using SYBR™ Green PCR Master Mix. A, a PCR reaction readout from the ABI7700 Real-Time Detection device. In this experiment, a PCR reaction was performed in triplicate samples. Notice that towards the end of the PCR reaction, a difference in amount of product produced is observed. B, the linearity of the plots shows the equal amplification of the assay over a range of input cDNA concentration. C, dissociation curves provide a graphical representation of the PCR product after the amplification process. A single peak in positive samples suggests a single size product. The melting temperature of each PCR product varies and is dependent on its sequence and size. D, three real-time amplification plots are shown.

FIG. 3. Quantitative analysis of OMD and PRELP gene expressions in bladder tissues using qRT-PCR. A, expression profile of OMD. Quantitative RT-PCR was used to study gene expression in a cohort of bladder cancers and normal bladder samples. Relative gene expression was assessed using the method of Pfaffl, a modified method of comparative quantification. B, OMD gene expression in normal and tumor tissues is shown by the box-whisker plot. P value was calculated using the Mann-Whitney U test. We evaluated the cutoff value as follows: Cutoff (OMD)=[(smallest non-outlier observation in normal bladder tissues)+(largest non-outlier observation in tumor bladder tissues)]/2 C, expression profile of PRELP. Quantitative RT-PCR was used to study gene expression in a cohort of bladder cancers and normal bladder samples. Relative gene expression was assessed using the method of Pfaffl, a modified method of comparative quantification. D, PRELP gene expression in normal and tumor tissues is shown by the box-whisker plot. P value was calculated using the Mann-Whitney U test. We evaluated cut-off value as indicated above.

FIG. 4. Quantitative analysis of OMD and PRELP gene expressions in renal tissues using qRT-PCR. A, expression profile of OMD. Quantitative RT-PCR was used to study gene expression in a cohort of bladder cancers and normal bladder samples. Relative gene expression was assessed using the method of Pfaffl, a modified method of comparative quantification. B, OMD gene expression in normal and tumor tissues is shown by the box-whisker plot. P value indicated in FIG. 3. C, expression profile of PRELP. Quantitative RT-PCR was used to study gene expression in a cohort of bladder cancers and normal bladder samples. Relative gene expression was assessed using the method of Pfaffl, a modified method of comparative quantification. D, the PRELP gene expression in normal and tumor tissues are shown by the box-whisker plot. P value was calculated using Mann-Whitney U test. We evaluated cutoff value as indicated in FIG. 3.

FIG. 5. Quantitative analysis of OMD and PRELP gene expression in several normal tissues, bladder tumor tissues and bladder cancer cell lines using qRT-PCR. A, Relative gene expression of OMD in nine normal tissues and bladder cancer tissues. B, Relative gene expression of osteomodulin in 10 bladder cancer cell lines, and bladder tissues (normal and tumor). C, Relative gene expression of PRELP in 9 normal tissues and bladder tumor tissues. D, Relative gene expression of PRELP in 10 bladder cancer cell lines, and bladder tissues (normal and tumor).

FIG. 6. Quantitative analysis of OMD gene expression in various types of cancer using microarray. OMD gene expression profiles as Dot-Box analysis were obtained by using gene expression profiling data. In each case, OMD expression in the corresponding normal tissues is indicated first and then OMD expression in the described cancer tissues is indicated by yellow boxes.

FIG. 7. Quantitative analysis of PRELP gene expression in various types of cancer using microarray. PRELP gene expression profiles as Dot-Box analysis were obtained by using gene expression profiling data. In each case, PRELP expression in the corresponding normal tissues is indicated first and then PRELP expression in the described cancer tissues is indicated by yellow boxes.

FIG. 8. Distribution of PRELP protein in bladder normal tissues and cancer tissues. Immunohistochemistry using PRELP antibody (Panel A) or control IgG (Panel B) were performed using normal bladder and bladder cancer tissues. PRELP protein is observed in normal bladder tissues. However, PRELP protein is completely excluded in bladder cancer. Negative control (panel B) has no staining.

FIG. 9. Cells with abnormal shapes after overexpression of OMD in EJ28 bladder cancer cells. EJ28 bladder cancer cell line was stably transfected with OMD expression construct. This transfection increased number of apoptotic cell and the cells showed abnormal shapes.

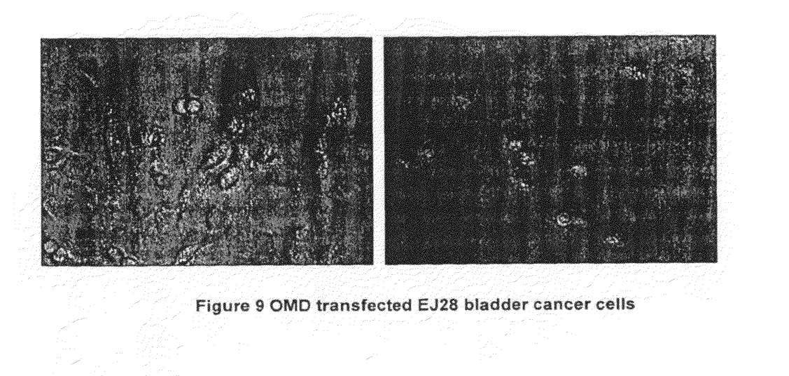

FIG. 10. OMD expression protects normal cells from apoptosis, whilst PRELP expression has no effect. HEK 293 cells stably transfected with either CAT (a control), OMD or PRELP, and assayed to measure the level of apoptosis they underwent in response to treatment with 1 ug/ml mitomycin C. (a) Annexin assay. Cells were treated one dose of the drug, and 24 hours later, they were trypsinized, incubated in the presence of Alexafluor-647 conjugated annexin and propidium iodide, and examined by flow cytometry. The annexin-positive, PI-negative subpopulation, comprising live cells that were in the process of undergoing apoptosis, was identified. (b) Caspase activity assay. One dose of the drug was administered, and 24 h later, cells were incubated in the presence of a substrate that, upon cleavage by caspases, was converted into a luminescent product. Luminescence was quantified and taken to be proportional to caspase activity. In both (a) and (b), error bars refer to standard deviations, and statistical analysis consisted of t-tests.

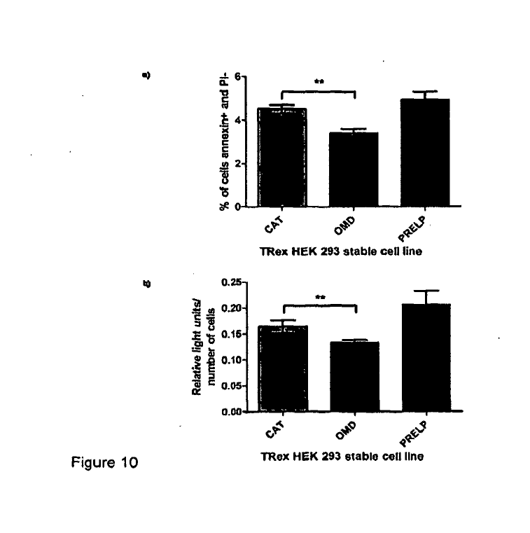

FIG. 11. Overexpression of OMD or PRELP in EJ28 cells results in sensitization of the cells to Mitomycin C treatment.

Two control EJ28 cells, two OMD stably-transfected EJ28 cells, and a PRELP stably-transfected EJ28 cells are treated with 1 μg/ml of Mitomycin C. Also, as a positive control, EJ28 cells are treated with higher concentration 5 μg/ml of Mitomycin C as a positive control. Then, the ratios of apoptotic cells were determined by measuring caspase activities.

FIG. 12. Overexpressed proteins of OMD and PRELP

The overexpressed proteins of OMD and PRELP were confirmed by western blotting.

FIG. 13. Effect of siPRELP transfection with 5637 bladder cancer cell line A. After transfection of siPRELP with 5637 bladder cancer cell line, its effect on PRELP mRNA level was examined. B-F. Our microarray analysis using siPRELP with 5637 bladder cancer cell line has identified many significantly modified genes (see Table 6). The result of microarray data was confirmed quantitative RT-PCR of several selected genes. B, ZMAT3, C, CASP3, D, CSNK1A1, E, PPP2R1B, F, DNMT1.

FIG. 14. OMD abolishes, and PRELP inhibits, anchorage-independent growth of EJ28 cells. Cells were seeded in DMEM+0.3% agar, overlying a lower layer of DMEM+0.6% agar. 3000 cells were seeded into wells of a 6-well dish in triplicate. Plates were incubated for 2 weeks, and colonies were counted. Error bars are standard deviations. Statistical analysis consisted of one-way ANOVA, with post-hoc Newman-Keuls testing. Letter groupings, “a”, “b” etc, refer to the results of the Newman-Keuls test. Cell lines not significantly different to each other are labelled with the same letter. Cell lines that are significantly different to each other (p<0.05) are labelled with different letters.

FIG. 15 Effect of xenograft of EJ28 cells overexpressing OMD protein. EJ28 bladder cancer cells or EJ28 cells overexpressing OMD were inoculated into nude mice and then cancer development was monitored for three weeks. The result at 18 days is shown. The control mice inoculated by EJ28 cells developed significant cancer, while the mice with OMD expressing cells did not develop any cancer.

### EXAMPLES

### Example 1

### Background to SLRPS

OMD and PRELP are members of the small leucine-rich repeat proteoglycans (SLRP) family of proteins which are present in extracellular matrices.

The extracellular matrix (ECM) is believed to play an important role in the regulation of tumour initiation and growth through regulation of microenvironment. Normal cells require a basement membrane for growth. With the development of epithelial malignancy, major changes occur in the organization and distribution of ECM, which supports and forms the basement membrane (BM). Invasive tumors are characterized by a defective BM adjacent to cells, whereas in benign tumors the BM remains intact (Liotta, 1986).

The SLRP family is characterized by the conserved leucine rich repeat domain at the centre of proteins. The number of repeats depends on the members. The SLRP family members have significantly distinct the NH₂-termini and COOH-termini, which largely provides the functional differences between these proteins. The N-terminal and C-terminal regions of many members have important cysteine residues. Ten of the 16 known SLRP genes are arranged in tandem clusters on human chromosome 1, 9, and 12 and have syntenic equivalents in rat and mouse. Also, these proteins have sugar modifications. However, each member has a distinct type of sugar modification.

They are also functionally important for the integration of signaling pathways in the ECM (Hocking et al., 1998; Kuriyama et al., 2006; Ohta et al., 2006; Ohta et al., 2004). Members of the SLRP family bind a variety of extracellular proteins including growth factors, signaling ligands and ECM components and regulate a variety of biological events. These events include ligand induced signaling. They regulate many ligand-induced signaling pathways through direct interaction with their extracellular signaling components. Different SLRPs regulate different pathways and different biological events. Tsukushi regulates the BMP, nodal, FGF, and Notch pathways (Kuriyama et al., 2006; Morris et al., 2007; Ohta et al., 2006; Ohta et al., 2004), while decorin regulates the EGF and TGF-beta pathways (Patel et al., 1998; Takeuchi et al., 1994). Also, through interactions with ECM proteins including type I collagen (Hedbom and Heinegard, 1993; Rada et al., 1993; Schonherr et al., 1995; Vogel et al., 1984) they are thought to guide matrix assembly and organization through protein-protein and/or protein-carbohydrate interactions. Different SLRPs affect the fibril formation of collagen: in vitro, the interaction of decorin, fibromodulin and lumican with fibrillar collagens alters fibril size by slowing the rate of fibril formation and influencing collagen fibril diameter. SLRPs are localized in different tissue types (Alimohamad et al., 2005), and collagen deposition varies between tissues, so SLRPs it is possible directly affect ECM organization.

Reflecting the variety of their activities, mutations of these proteoglycans are known to results in distinct human disorders. For example, nyctalpin (Bech-Hansen et al., 2000; Pusch et al., 2000) mutation is known to be associated with night blindness. Asporin is involved in osteoarthritis (Kizawa et al., 2005). Mice deficient in decorin, fibromodulin, keratocan and lumican-deficient exhibit numerous abnormalities in the arrangement and structure of collagen fibrils in skin, tendon, cornea, and sclera (Austin et al., 2002; Danielson et al., 1997; Liu et al., 2003; Svensson et al., 1999). Moreover, mutations in the keratocan gene have been shown to cause a severe recessively inherited form of cornea plana in humans, a condition characterized by corneal flattening and reduction of refractive power of the cornea (Pellegata et al., 2000). SLRPs also form functionally important complexes with numerous signaling molecules. These observations indicate that functions of SLRP family members are diverse.

Furthermore, the expression of SLRPs in cancer varies, depending on the family member in question and the type of cancer. For example, mRNA of TSK is increased in breast and lung cancers (see WO2004035627) lumican is overexpressed in some cancer types studied [breast (Leygue et al., 1998), cervix (Naito et al., 2002), pancreas and colon (Lu et al., 2002)]. Decorin is overexpressed in breast cancer (Leygue et al., 2000) and leukaemia (Campo et al., 2006), but underexpressed in thyroid cancer (Arnaldi et al., 2005) and ovarian tumours (Nash et al., 2002). Biglycan is overexpressed in pancreatic cancer (Weber et al., 2001). Also, in the case of function, decorin and lumican was suggested to have tumour-suppressing activity in some cancer types, while TSK is oncogenic. In breast cancer, lumican expression correlates with tumor grade, estrogen levels and age of patients (Leygue et al., 1998). Decorin/p53 double knockout mice almost uniformly develop thymic lymphoma (Iozzo et al., 1999a), in contrast to decorin single knockout mice, which show no predisposition to cancer, and p53 single knockout mice, which are predisposed to an array of different cancers. It appears that lack of decorin accelerates carcinogenesis in a p53-deficient background. Functional analysis suggests that SLRPs can regulate a number of processes involved in carcinogenesis. Stable transfection of decorin suppressed xenografted cancer cells from forming tumors in mice (Santra et al., 1995), and suppressed the proliferation of squamous carcinoma cells in vitro by binding the EGFR, causing its autophosphorylation and triggering prolonged activation of the MAP kinase cascade and upregulation of p21 Cip1/WAF1 (Iozzo et al., 1999b; Moscatello et al., 1998). The treatment of xenografted cancer cells with exogenous decorin, or their stable transfection with lumican, reduced metastases in recipient mice (Reed et al., 2005; Vuillermoz et al., 2004), whilst mice constitutively overexpressing biglycan displayed elevated angiogenesis (Shimizu-Hirota et al., 2004).

Thus, although there is a precedent for certain SLRPs having a role in cancer, their precise role in human carcinogenesis has not been clear. OMD or PRELP expression patterns are quite different from other SLRPs. Also, OMD or PRELP regulates Wnt pathway and tight junction pathway, which was not reported as downstream of other SLRPs. Careful detailed analysis of each SLRP member in defined tumours is required to know their true function in tumourigenesis in the selected cancer.

### Example 2

### Examination of Diagnostic Value of OMD and PRELP in Bladder Cancer Using Quantitative RT-PCR

126 surgical specimens of primary urothelial cell carcinoma were collected, either at cystectomy or trans-uretheral resection, and snap frozen in liquid nitrogen. Thirty-four specimens of normal bladder urothelium were collected from areas of macroscopically normal urothelium in patients with no evidence of urothelial malignancy. Use of tissues for this study was approved by Cambridgeshire Local Research Ethics Committee.

Cancer tissues and normal tissues were isolated by laser capture microdissection by following the procedure. Five sequential sections of 7 μm thickness were cut from each tissue and stained using Histogene™ staining solution (Arcturus, Calif., USA) following the manufacturer's protocol. Slides were then immediately transferred for microdissection using a Pix Cell II laser capture microscope (Arcturus, Calif., USA). Two 7 μm ‘sandwich’ sections adjacent to the tissue used for RNA extraction were sectioned, stained and assessed for cellularity and tumor grade by an independent consultant urohistopathologist. Additionally, the sections were graded according to the degree of inflammatory cell infiltration (low, moderate and significant). Samples showing significant inflammatory cell infiltration were excluded (Wallard et al., 2006). Approximately 10,000 cells were microdissected from both stromal and epithelial/tumor compartments in each tissue. Tissues containing significant inflammatory cell infiltration were avoided to prevent contamination.

Total RNA was extracted using TRI Reagent™ (Sigma, Dorset, UK), following the manufacturers protocol. RNEasy Minikit™ (Qiagen, Crawley, UK), including a DNase step, was used to optimize RNA purity. Agilent 2100™ total RNA bioanalysis was performed. One microliter of resuspended RNA from each sample was applied to an RNA 6000 NanoLabChip™, and processed according to the manufacturer's instructions. All chips and reagents were sourced from Agilent Technologies™ (West Lothian, UK).

Total RNA concentrations were determined using the Nanodrop™ ND1000 spectrophotometer (Nyxor Biotech, Paris, France). The endogenous 18S CT value was used as an accurate measure of the amount of intact starting RNA. One microgram of total RNA was reverse transcribed with 2 μg random hexamers (Amersham) and Superscript III reverse transcriptase (Invitrogen, Paisley, UK) in 20 μl reactions according to the manufacturer's instructions. cDNA was then diluted 1:100 with PCR grade water and stored at −20° C.

For quantitative RT-PCR reactions, specific primers for all human GAPDH (housekeeping gene), SDH (housekeeping gene), OMD and PRELP were designed (Table 1). For 18S amplification, TaqMan Ribosomal RNA Control Reagents were purchased from Applied Biosystems, Warrington, UK. PCR reactions were performed using the ABI prism 7700 Sequence Detection System (Applied Biosystems, Warrington, UK) following the manufactures protocol. Reactions for 18S analyses were performed in 10 μl PCR volumes containing the equivalent of 1 ng of reverse transcribed RNA, 50% SYBR GREEN universal PCR Master Mix without UNG (Applied Biosystems, Warrington, UK), 200 nM each of the forward and reverse primers and 100 nM of probe. Amplification conditions were 2 min at 50° C., 10 min at 95° C. and then 40 cycles each consisting of 15 s at 95° C. and 1 min at 60° C. Reaction conditions for target gene amplification were as described above and the equivalent of 5 ng of reverse transcribed RNA was used in each reaction. To determine relative RNA levels within the samples, standard curves for the PCR reactions were prepared from a series of two-fold dilutions of cDNA covering the range 2-0.625 ng of RNA for the 18S reaction and 20-0.5 ng of RNA for all target genes. The ABI prism 7700 measured changes in fluorescence levels throughout the 40-cycle PCR reaction and generated a cycle threshold (CO value for each sample correlating to the point at which amplification entered the exponential phase. This value was used as an indicator of the amount of starting template; hence a lower Cₜ values indicated a higher amount of initial intact cDNA. To validate the accuracy of microdissection, primers and probes for Vimentin and Uroplakin were sourced and qRT-PCR performed according to the manufacturer's instructions (Assays on demand, Applied Biosystems, Warrington, UK). Vimentin is primarily expressed in messenchymally derived cells, and was used as a stromal marker. Uroplakin is a marker of urothelial differentiation and is preserved in up to 90% of epithelially derived tumors (Olsburgh et al., 2003).

RNA expression levels for each target gene were normalized to the endogenous 18S rRNA levels. For grade correlation studies, two-tailed Spearman's Rank Correlation was performed to determine the significance of the relationship between gene expression and increasing cancer grade. To determine the significance of differential expression in the laser captured tissue, a two-sided Mann-Whitney U nonparametric analysis was performed, for which a P-value of <0.05 was considered significant. Statistical evaluations were done using the STATA (version 8.0; StateCorp, College Station, Tex., USA) and StatView (version 5.0; SAS, Cary, N.C., USA).

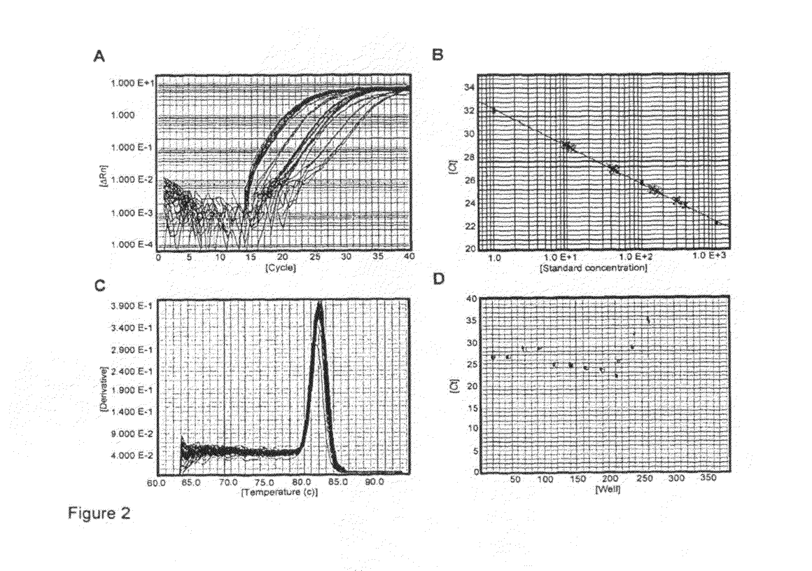

A real-time-PCR read-out is given as the number of PCR cycles (“cycle threshold” Ct) necessary to achieve a given level of fluorescence. For this study, the Ct was fixed in the exponential phase of the PCR (FIG. 2A, linear part of the fluorescence curve). During the initial PCR cycles, the fluorescence signal emitted by SYBR-Green I bound to PCR product was usually too weak to register above the background, and could not be defined until after about 15 PCR cycles. During the exponential phase of the PCR the fluorescence doubled at each cycle. After 35 cycles, the intensity of the fluorescent signal usually began to plateau, indicating that the PCR had reached a saturation status. As a Ct is proportional to the logarithm of initial amount of target in a sample, the relative concentration of one target with respect to another is reflected in the difference in cycle number (ΔCt) necessary to achieve the same level of fluorescence. Ct values at a fixed threshold of relative fluorescence were determined. Calibration curves were constructed by plotting Ct values as a function of log of total RNA, assuming that RNA targets were reversed, transcribed, and subsequently amplified with similar efficiency (FIG. 2B). Analysis of the melting curve profiles confirmed the specific accumulation of the amplification products (FIG. 2C). Data were obtained from triplicate assays, and each replicate datum is was always very similar (FIG. 2D).

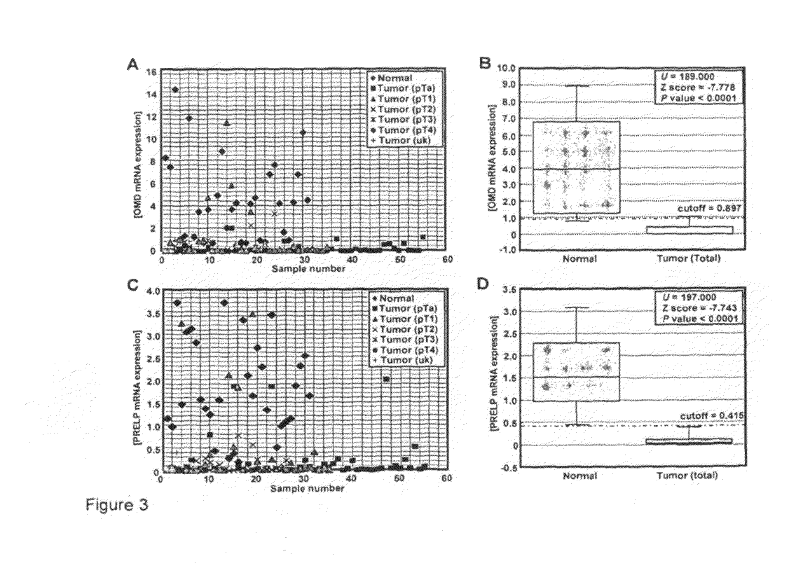

The results are indicated in FIG. 3 and Table 2 and summarised hereinbefore.

### Example 3

### Examination of Diagnostic Value of OMD and PRELP in Kidney Cancer by Quantitative RT-PCR

78 renal cell carcinoma surgical specimens of primary kidney carcinoma were collected and snap frozen in liquid nitrogen. 15vspecimens of normal kidney urothelium were collected from areas of macroscopically normal urothelium in patients with no evidence of urothelial malignancy. Use of tissues for this study was approved by Cambridgeshire Local Research Ethics Committee. All further procedures for quantitative RT-PCR were performed as described above.

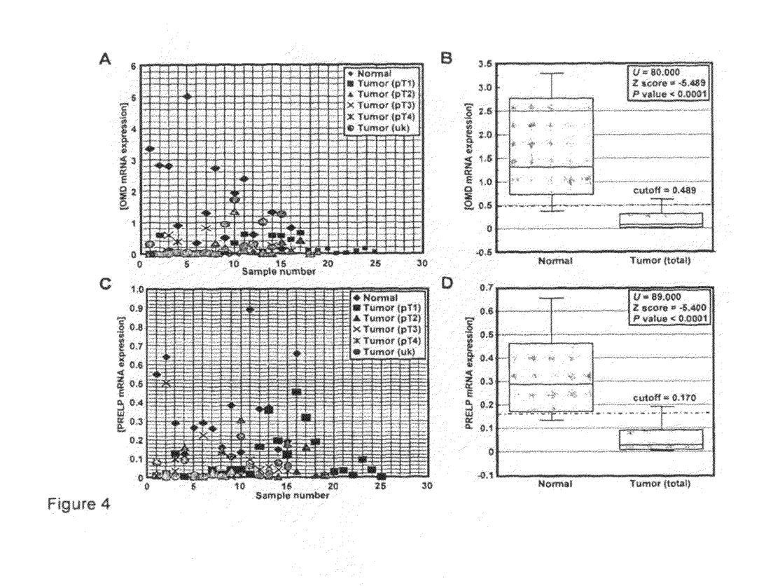

The results are indicated in FIG. 4 and Table 3 and summarised in the main text. Based on FIGS. 3 and 4, Table 3 and 4, we examined diagnostic values of OMD and PRELP (Table 4). The result is summarised hereinbefore.

### Example 4

### Expression of OMD and PRELP in Cancer Cell Lines and Normal Human Tissues

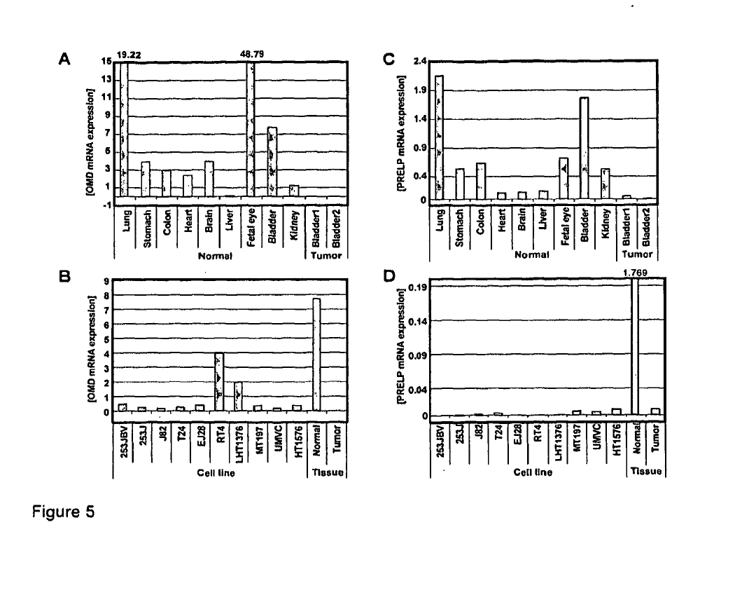

RNAs were isolated from normal human tissues of lung, stomach, colon, heart, brain, liver, eye, bladder and kidney. Also, RNAs were isolated from two bladder cancer samples. Then, quantitative RT-PCR was performed as described above using OMD and PRELP primers (Table 1). FIG. 5A shows that OMD is most highly expressed in eye and lung. Also, a significant amount of expression was observed in all other tissues, except liver. On the other hand, PRELP is highly expressed in lung and bladder. All other tissues including liver have a significant expression (FIG. 5C). The Cancer cell lines, 253JBV, 253J, J82, T24, EJ28, RT4, LHT1376, MT197, UMVC, and HT1576, were cultured and then total RNAs were isolated as described. RNAs from normal bladder and bladder cancer were used as control. Expression of OMD and PRELP in the cancer cell lines was determined by quantitative RT-PCR as described. Expression of OMD was strongly suppressed in all bladder cancer cell lines except RT4 and LHT1376 (FIG. 5B). This is consistent with our expression analysis as shown in Table 3. Expression level of OMD has correlation with stage of cancer. These cell lines are known as well-differentiated low-grade bladder cell lines. In the case of PRELP, its expression was almost completely suppressed in all cell lines examined (FIG. 5D).

### Example 5

### Examination of Diagnostic Value of OMD in Various Types of Cancer

As shown above, OMD gene expression is very strongly suppressed in bladder and kidney cancers. To examine OMD gene expression in various types of malignant and normal human tissues, we used the gene expression database based on microarray analysis using mRNA isolated from tumors and corresponding normal tissues from a large number of human patients (Gene Logic Inc. (Gaithersburg, Md.). RNA was prepared and gene expression analysis was determined at Gene Logic Inc. using Affymetrix GeneChip® HG-U133Plus2 microarrays containing oligodeoxynucleotides that correspond to approximately 40,000 genes/ESTs. We showed OMD gene expression profiles as Dot-Box analysis in house by using gene expression profiling data and accompanying clinical data purchased from GeneLogic Inc.

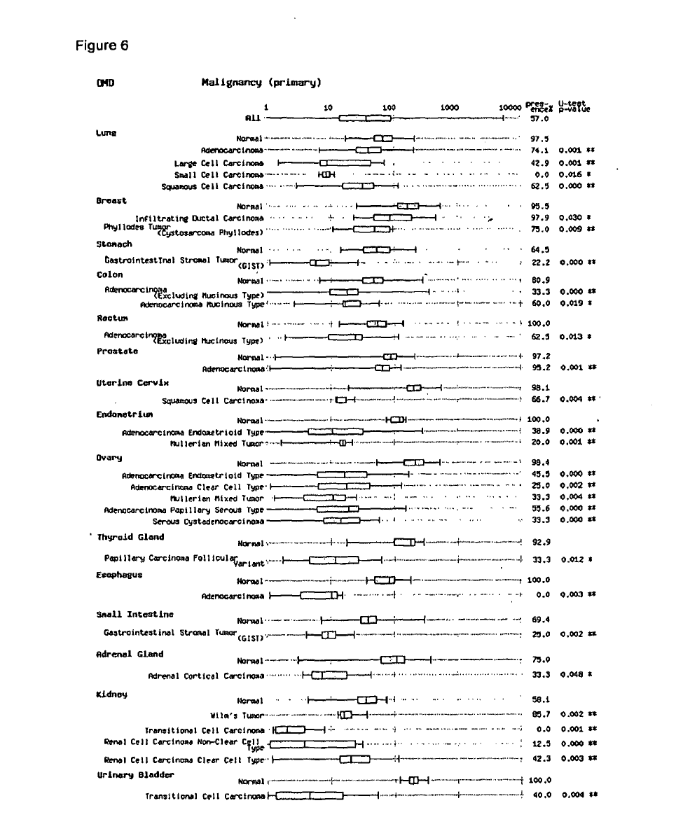

As shown in FIG. 6, the OMD expression is significantly downregulated in lung cancer (adenocarcinoma, large cell carcinoma, small cell carcinoma, squamous cell carcinoma), breast cancer (infiltrating ductal carcinoma and phyllodes tumour), stomach cancer (gastrointestinal storomal tumour). Colon cancer (adenocarcinoma), Rectum cancer (adenocarcinoma), Prostate cancer (adenocarcinoma), Utrine cervix cancer (Squamous cell carcinoma), Endometrium cancer (adenocarcinoma endometrioid type, Mullerian mixed tumour), Ovary cancer (adenocarcinoma endometrioid type, adenocarcinoma clear cell type, Mullerian mixed tumour, adenocarcinoma papillary serous type, serous cystadenocarcinoma), Thyroid grand (papillary carcinoma), Esophagus cancer (adenocarcinoma), Small intestine (gastrointestinal stromal tumour), Adrenal gland (adrenal cortical carcinoma), Kidney cancer (Wilm's tumour, transitional cell carcinoma, renal cell carcinoma), and Urinary bladder cancer (transitional cell carcinoma). These observations indicate that OMD is functional as a marker of various types of cancers.

### Example 6

### Examination of Diagnostic Value of PRELP in Various Types of Cancer

As shown above, PRELP gene expression is very strongly suppressed in bladder and kidney cancers. To examine PRELP gene expression in various types of malignant and normal human tissues, we used the gene expression database based on microarray analysis using mRNA isolated from tumors and corresponding normal tissues from a large number of human patients (Gene Logic Inc. (Gaithersburg, Md.). RNA was prepared and gene expression analysis was determined at Gene Logic Inc. using Affymetrix GeneChip® HG-U133Plus2 microarrays containing oligodeoxynucleotides that correspond to approximately 40,000 genes/ESTs. We showed PRELP gene expression profiles as Dot-Box analysis in house by using gene expression profiling data and accompanying clinical data purchased from GeneLogic Inc.

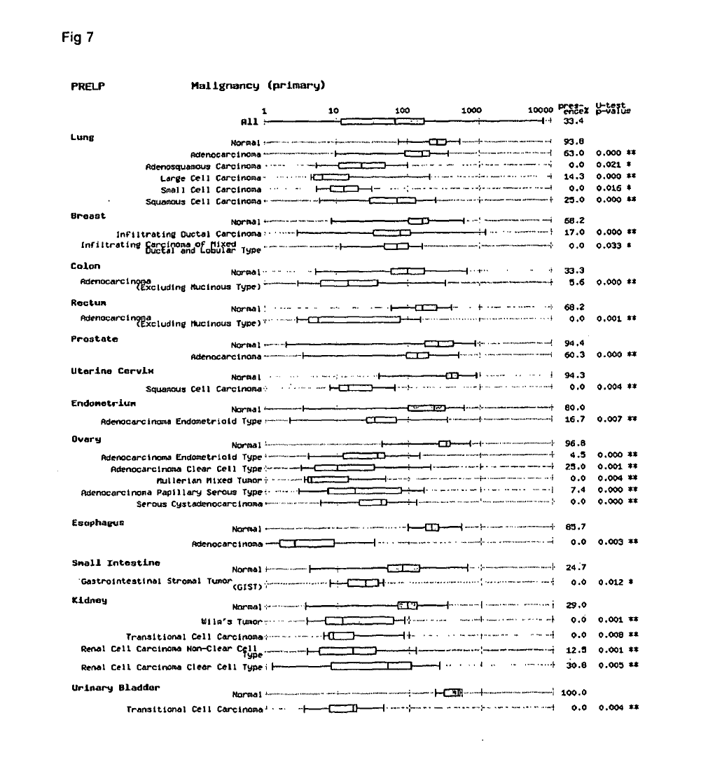

As shown in FIG. 7, the PRELP expression is significantly downregulated in lung cancer (Adenocarcinoma, adenosquamous carcinoma, large cell carcinoma, small cell carcinoma, squamous cell carcinoma), breast cancer (infiltrating ductal carcinoma and infiltrating carcinoma of mixed ductal and lobular type), Colon cancer (adenocarcinoma), Rectum cancer (adenocarcinoma), Prostate cancer (adenocarcinoma), Utrine cervix cancer (Squamous cell carcinoma), Endometrium cancer (adenocarcinoma endometrioid type), Ovary cancer (adenocarcinoma endometrioid type, adenocarcinoma clear cell type, Mullerian mixed tumour, adenocarcinoma papillary serous type, serous cystadenocarcinoma), Esophagus cancer (adenocarcinoma), Small intestine (gastrointestinal stromal tumour), Kidney cancer (Wilm's tumour, transitional cell carcinoma, renal cell carcinoma), and Urinary bladder cancer (transitional cell carcinoma). These observations indicate that PRELP is functional as a marker of various types of cancers.

### Example 7

### PRELP Protein Distribution in Bladder Normal Tissues and Cancer Tissues

To confirm diagnostic value of PRELP, the protein expression of PRELP was examined by immunohistochemostry using a PRELP antibody and bladder cancer tissues. Frozen section were prepared from fresh human normal bladder and bladder cancer and fixed in 4% paraformaldehyde in PBS, for 15 min at RT. Then, the sections were washed in PBS(−), 5 min×3 and treated with 0.3% Hydrogen Peroxide in methanol for 15 min at RT. The slides were washed in PBS(−), 5 min×3 and blocked in 3% BSA in PBS(−). Then, 1^{st} antibody (1/500 diluted anti-PRELP (mouse polyclonal, cat#: H00005519-B01, Abnova) and normal mouse IgG (sc-2050, SantaCruz) in Blocking Soln) was applied to the slides and incubated overnight at 4° C. The sides were washed in PBS (−) 5 min×3 and incubated with 2^{nd} antibody (1/500 diluted antibody in Blocking soln) for 30 min at RT. The slides were washed in PBS (−) 5 min×3 and treated with ABC reagent (Vector) for 30 min at RT. After washing in PBS (−) 5 min×3, the slides were incubated with DAB substrate kit (Vector) at RT, under observation. At suitable staining, the reaction was stopped by excess DDW. The slides were dehydrated by ethanol and Xylene and mount in VectaMount.

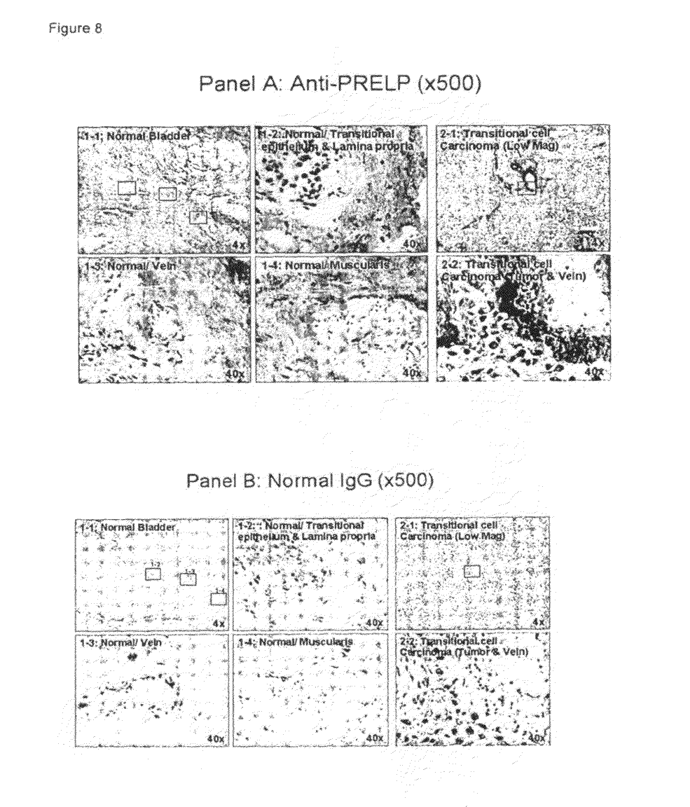

PRELP protein is widely expressed in normal bladder tissues especially in stroma. On the other hand, PRELP protein staining is almost completed excluded in bladder cancer tissues (FIG. 8). This observation is consistent with our analysis of PRELP mRNA in bladder cancer tissues and support our invention about the value of PRELP in bladder cancer diagnosis.

### Example 8

### Effect of OMD on Cancer Cells

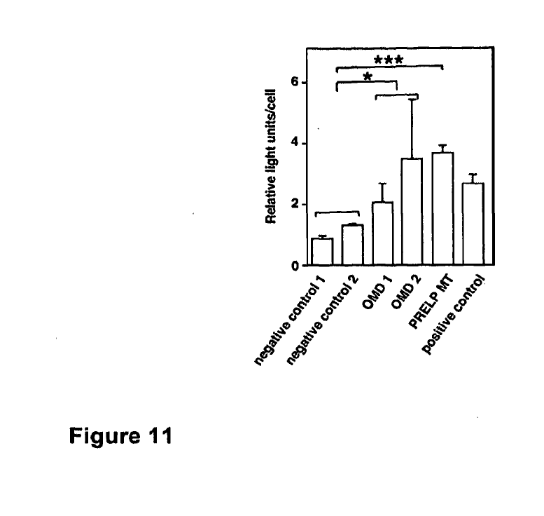

OMD and PRELP were subcloned into pIRES2-EGFP (Clontech). A bladder cancer cell line, EJ28, was stably transfected with these plasmids by selection with G418. Two independent clones were derived for each plasmid (except MT-Prelp, where it was only possible to derive one). Then, the properties of these cells were examined. OMD-transfected and Myc-tagged OMD-transfected cells displayed an unusual morphology; when in low confluence, they were rounded up with actively blebbing cell membranes, suggesting a problem with cellular adhesion. These cells include apoptotic ones. This contrasted with control cells, which were flat and cuboidal. OMD-transfected cells proliferated more slowly than control cells. This was demonstrated by slower proliferation in a cell-counting assay and a lower rate of BrdU incorporation. OMD-transfected cells also displayed a lower proportion of cells in S-phase as measured in FACS analysis. They were markedly sensitized to apoptosis induced by Mitomycin C, a drug used in the treatment of early bladder cancer (FIG. 11). Two independent clones of OMD and EGFP expressing EJ28 cells, two independent control EJ28 clones expressing EGFP, and a PRELP with myc tag and EGFP expressing EJ28 cells were treated with 1 μg/ml Mitomycin C. Also, EGFP expressing EJ28 cells were treated with higher concentration of Mitomycin C (5 μg/ml) as a positive control. In the positive control, massive cell death was observed.8As indicated in FIG. 11, OMD expressed cells showed activated apoptosis, indicating that OMD overexpression sensitizes cells to Mitomycin C mediated cell death. Also, PRELP expressing cells also showed altered properties. They displayed higher rates of endogenous apoptosis, and displayed even higher rates of apoptosis in response to treatment with Mitomycin C (FIG. 11) although cell cycle inhibition was not observed

### Example 9

### OMD or PRELP Selectively Kill Transformed Cancer Cells

OMD and PRELP have the ability to kill cancer cells and potentiate cancer drug mediated cell death. Interestingly, this chemosensitization was unique to cancer cells. OMD overexpression actually protected normal cells from Mitomycin-C mediated apoptosis whilst PRELP had no effect on their sensitivity (FIG. 11). This suggests that treatment with OMD and/or PRELP, in combination with Mitomycin C treatment, would enhance killing of cancer cells, but protect normal cells.

### Example 10

### OMD Abolishes and PRELP Reduces Anchorage-Independent Growth of Cancer Cells

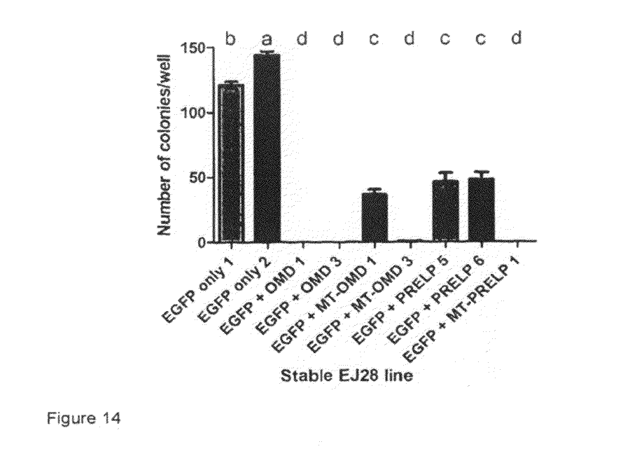

OMD and PRELP also affect the anchorage-independence, a hallmark of cancer cells. Anchorage-independence was measured by seeding cells in soft agar, incubating them for 2 weeks and counting the number of resultant colonies. Strikingly, OMD overexpression absolutely abolished anchorage-independence of EJ28 cells, suggesting that OMD could dramatically inhibit tumour formation. PRELP also inhibits anchorage-independent growth of EJ28, and reduces colony-forming ability in soft agar to a third of that observed in control cells (FIG. 14).

### Example 11

### Examination of Molecular Mechanisms of OMD or PRELP Mediated Cancer Cell Death

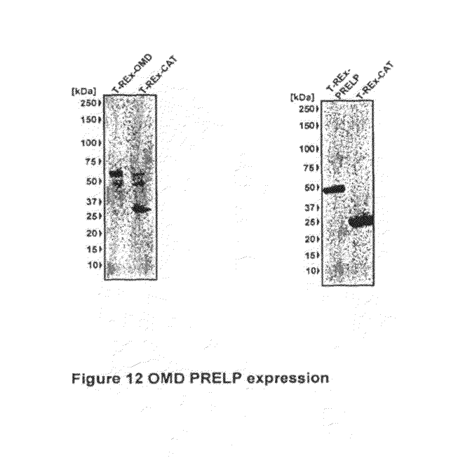

To determine the mechanism of how activation of OMD or PRELP kills cancer cells, two types cells were constructed: OMD or PRELP expressing cells and OMD or PRELP deleted cells. To overexpress the genes, the T-Rex-293 system was used according to the manufacture's instruction. This system enables the expression of target proteins without influencing expression of endogenous proteins. In brief, 293 cells were transfected with pcDNA5-FRT/TO-OMD or pcDNA5-FRT/TO-PRELP using lipofectamine 2000. Stablly transformed cells were selected and then three independent colonies were isolated. After confirmation of identical expression levels of OMD or PRELP in these cell lines (FIG. 12), a cell line was used for further analysis.

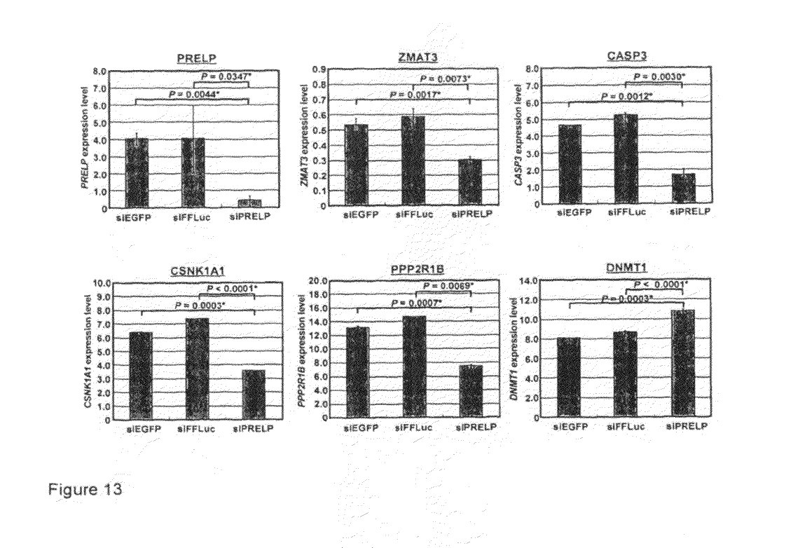

To delete OMD or PRELP expression, firstly we searched a suitable cell line because in almost all cancer cell lines their expressions are largely already suppressed. Our search identified 5637 bladder cancer cell line, which has some expressions although their expression levels are lower than those in normal tissues. The 5637-cell line was transfected with siOMD, siPRELP, siEGFP, or siFFLuc. Suppression of OMD or PRELP level was confirmed by quantitative RT-PCR as indicated in FIG. 13.

To determine molecular activity of OMD and PRELP in cancer development, downstream target genes and signaling pathways were determined by mRNA profiling using microarray. To this end, after culturing the cells, total RNA was isolated as described above. The total RNA was labeled and hybridized onto Affymetrix U133 Plus 2.0 GeneChip oligonucleotide arrays (Affymetrix) according to the manufacturer's instructions. Briefly, hybridization signals were scaled in the Affymetrix GCOS software (version 1.1.1) using a scaling factor determined by adjusting the global trimmed mean signal intensity value to 500 for each array and imported into GeneSpring version 6.2 (Silicon Genetics). Signal intensities were then centered to the 50^{th} percentile of each chip and, for each individual gene, to the median intensity of each specific subset first to minimize the possible technical bias and then to the whole sample set. The intensity of any replicate hybridisations was averaged subsequent to further analysis. Only genes labeled by the GCOS software as “present” or “marginal” in all samples were used for further analysis. Differentially expressed genes were identified using the Wilcoxon-Mann-Whitney nonparametric test (P<0.05). The Benjamini-Hochberg false discovery rate multiple test correction was applied whenever applicable. Hierarchical cluster analysis was done on each comparison to assess correlations among samples for each identified gene set.

Tables 5 and 6 show genes showing that their expressions are significantly and consistently up or downregulated by activation and suppression of OMD and PRELP. These include many oncogenes and tumour suppressor genes. To determine signalling pathways influenced by OMD and PRELP, we analysed the genes by the KEGG pathway analysis programme. Table 7 shows that the p53 pathway is the common main downstream pathway of OMD and PRELP. The p53 pathway is the most well established signaling pathway in tumourigenesis. In particular, mutation of p53 is known to be associated with a large number of cancer. However, the mutation cannot explain all cases of tumourigenesis. Also, loss of heterozygosity on chromosome 17 occurs during the late stages of urothelial carcinomas although expression of p53 is significantly suppressed in a certain population of cancer from early stages. This is a major difference from OMD and PRELP, which expression is almost completely suppressed in almost all cancers. Our results suggest that suppression of both OMD and PRELP has a significant contribution of suppression of the p53 pathway. Also, both genes regulate the tight junction and the apoptosis pathways. The tight junction is known to regulate initial step of tumorigenesis, escape from anchorage-dependent growth (Tsukita et al., 2008). The apoptosis pathway is well known to be important for tumourigenesis (Brown and Attardi, 2005; Fesik, 2005; Johnstone et al., 2002; Li et al., 2008; Vazquez et al., 2008; Yu and Zhang, 2004). In addition, OMD regulates the Wnt pathway, which is also known to be involved in early stages of tumourigenesis (Bienz and Clevers, 2000; Clevers, 2004; Polakis, 2000; Reya and Clevers, 2005; Taipale and Beachy, 2001), and the adherens junction pathway, which is important for tumourigenesis (Giehl and Menke, 2008). These observations indicate that OMD and PRELP are largely functionally complementary but not completely redundant. Also, the influenced pathway is significantly different from other members of the SLRP family such as Tsukushi and decorin. These analyses indicate that OMD and PRELP kills cancer cells through activation of multiple tumour suppressing signals including the p53 pathway, the tight junction pathway and the apoptotic pathway. This clearly demonstrates the value of this invention for treatment of cancer.

### Example 12

### Evaluation of Therapeutic Potential of OMD Using Mouse Xenograft Models

Six- to eight-week-old male MF1 nude mice were obtained from Royal Free Hospital London UK. Tumors were induced by inoculation of 5×106 EJ28 cancer bladder cells s.c. on the back. EJ28 tumour-bearing MF-1 mice (n=5) were injected with cells subcloned with stably transfected with OMD-myc tag in pIRES2-EGFP vector, using Lipofectamine 2000 (Invitrogen). For the control, EJ28 cells were transfected with the vector without the overexpressed OMD gene. Tumour dimensions were measured continuously using a caliper and tumour volumes were calculated using the equation: volume=(π/6)×a×b×c, where a, b, and c represent three orthogonal axes of the tumour. Animals were assessed for tumour growth over 25-day period.

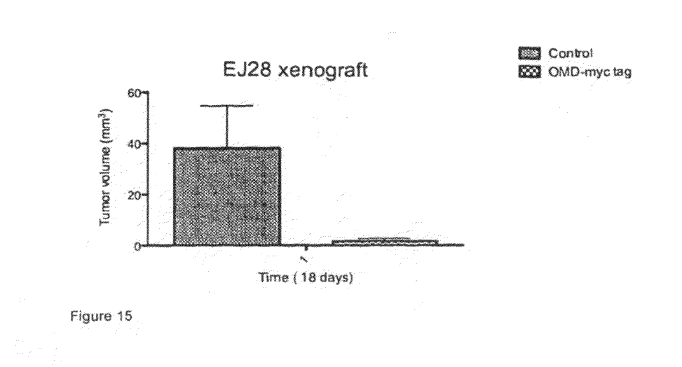

The biological activity of OMD on EJ28 tumour-bearing mice was determined by measuring changes in tumour volume. FIG. 15 shows the growth characteristics of EJ28 tumours in MF-1 mice injected with the OMD-myc-tag compared to the control. The results obtained showed a significant growth arrest of the tumor xenograft with OMD-myc tag compared to the control cells without the OMD gene. At day 18, tumour size for control was 37.91±16.57 mm³, (n=5), compared to the OMD-myc tag transfected cell line where tumour growth was completely inhibited (1.60±1.0 mm³).

TABLE 1
Primer sequences for quantitative RT-PCR

| Gene name | Primer sequence |
|---|---|
| GAPDH (housekeeping | 5′ GCAAATTCCATGGCACCGTC 3′ |
| gene) - f | |
| GAPDH (housekeeping | 5′ TCGCCCCACTTGATTTTGG 3′ |
| gene) - r | |
| SDH (housekeeping | 5′ TGGGAACAAGAGGGCATCTG 3′ |
| gene) - f | |
| SDH (housekeeping | 5′ CCACCACTGCATCAAATTCATG 3′ |
| gene) - r | |
| OMD - f | 5′ GCAAATTCCATGGCACCGTC 3′ |
| OMD - r | 5′ TCGCCCCACTTGATTTTGG 3′ |
| PRELP - f | 5′ CTGTCCCACAACAGGATCAGCAG 3′ |
| PRELP - r | 5′ CAGGTCCGAGGAGAAGTCATGG 3′ |

TABLE 2
Statistical analysis of OMD and PRELP expression levels in clinical bladder tissues.

| | OMD | | | | PRELP | | | |
|---|---|---|---|---|---|---|---|---|
| Characteristic | n | Mean | SD | 95% CI | n | Mean | SD | 95% CI |
| Normal (Control) | 31 | 4.398 | 3.605 | 3.076-5.721 | 31 | 1.674 | 0.939 | 1.324-2.025 |
| Tumor (Total) | 126 | 0.420 | 1.290 | 0.193-0.648 | 126 | 0.215 | 0.557 | 0.127-0.407 |
| Tumor stage | | | | | | | | |
| pTa, pT1 | 90 | 0.452 | 1.466 | 0.145-0.759 | 90 | 0.259 | 0.647 | 0.124-0.395 |
| pT2 | 26 | 0.433 | 0.772 | 0.121-0.745 | 26 | 0.121 | 0.183 | 0.047-0.195 |
| pT3, pT4 | 7 | 0.008 | 0.022 | −0.012-0.028 | 7 | 0.024 | 0.033 | −0.006-0.054 |
| Tumor grade | | | | | | | | |
| G1 | 12 | 1.127 | 3.267 | −0.948-3.203 | 10 | 0.498 | 0.873 | −0.057-1.053 |
| G2 | 63 | 0.280 | 0.763 | 0.088-0.472 | 63 | 0.210 | 0.635 | 0.050-0.370 |
| G3 | 50 | 0.436 | 0.981 | 0.157-0.715 | 50 | 0.157 | 0.297 | 0.072-0.241 |
| Metastasis | | | | | | | | |
| Negative | 99 | 0.484 | 1.430 | 0.197-0.771 | 99 | 0.252 | 0.619 | 0.128-0.375 |
| Positive | 27 | 0.185 | 0.352 | 0.062-0.334 | 27 | 0.080 | 0.163 | 0.015-0.144 |
| Gender | | | | | | | | |
| Male | 91 | 0.496 | 1.467 | 0.191-0.802 | 91 | 0.225 | 0.548 | 0.110-0.339 |
| Female | 32 | 0.244 | 0.633 | 0.015-0.472 | 32 | 0.179 | 0.614 | −0.042-0.401 |
| Recurrence | | | | | | | | |
| No | 28 | 0.167 | 0.447 | −0.007-0.340 | 28 | 0.137 | 0.350 | 0.001-0.273 |
| Yes | 51 | 0.384 | 1.122 | 0.069-0.700 | 51 | 0.145 | 0.375 | 0.039-0.250 |
| Died | 8 | 0.141 | 0.271 | −0.086-0.367 | 8 | 0.076 | 0.085 | 0.003-0.146 |

TABLE 3
Statistical analysis of OMD and PRELP expression levels in clinical renal tissues.

| | OMD | | | | PRELP | | | |
|---|---|---|---|---|---|---|---|---|
| Characteristic | n | Mean | SD | 95% CI | n | Mean | SD | 95% CI |
| Normal (Control) | 16 | 1.756 | 1.332 | 1.046-2.466 | 16 | 0.356 | 0.222 | 0.238-0.474 |
| Tumor (Total) | 79 | 0.220 | 0.340 | 0.144-0.296 | 79 | 0.070 | 0.102 | 0.047-0.093 |
| Tumor stage | | | | | | | | |
| pT1 | 25 | 0.202 | 0.238 | 0.104-0.301 | 25 | 0.093 | 0.123 | 0.042-0.143 |
| pT2 | 20 | 0.170 | 0.316 | 0.017-0.322 | 20 | 0.062 | 0.084 | 0.022-0.102 |
| pT3, pT4 | 19 | 0.145 | 0.127 | 0.043-0.248 | 19 | 0.064 | 0.116 | 0.010-0.118 |
| Tumor grade | | | | | | | | |
| G1, G2 | 42 | 0.240 | 0.388 | 0.119-0.361 | 42 | 0.068 | 0.098 | 0.038-0.099 |
| G3, G4 | 27 | 0.180 | 0.228 | 0.090-0.270 | 27 | 0.082 | 0.120 | 0.034-0.130 |
| Metastasis | | | | | | | | |
| Negative | 58 | 0.192 | 0.289 | 0.116-0.268 | 58 | 0.081 | 0.113 | 0.052-0.111 |
| Positive | 8 | 0.136 | 0.234 | −0.034-0.431 | 8 | 0.024 | 0.030 | −0.001-0.049 |
| Survival duration, yr | | | | | | | | |
| More than 5 | 46 | 0.187 | 0.264 | 0.108-0.265 | 46 | 0.068 | 0.098 | 0.039-0.097 |
| Less than 5 | 18 | 0.172 | 0.336 | 0.005-0.290 | 18 | 0.099 | 0.133 | 0.033-0.166 |
| Cambridge histology | | | | | | | | |
| Clear cell | 57 | 0.232 | 0.354 | 0.138-0.326 | 57 | 0.078 | 0.103 | 0.050-0.105 |
| Oncocytoma | 6 | 0.305 | 0.473 | −0.191-0.801 | 6 | 0.036 | 0.028 | 0.007-0.066 |
| TCC upper tract | 12 | 0.143 | 0.212 | 0.008-0.277 | 12 | 0.055 | 0.125 | −0.025-0.134 |
| Papillary | 2 | 0.304 | 0.403 | −3.317-3.925 | 2 | 0.099 | 0.134 | −1.106-1.304 |

TABLE 4
Relationship between OMD and PRELP expression levels and carcinogenesis

| | | | | | | | Tumor (Advanced | |
|---|---|---|---|---|---|---|---|---|
| | Normal | | Tumor (Total) | | Tumor (Early stage*) | | and late stages**) | |
| Characteristic | n | Specificity (%) | n | Sensitivity (%) | n | Sensitivity (%) | n | Sensitivity (%) |
| Bladder | | | | | | | | |
| OMD (cutoff: 0.897) | | | | | | | | |
| Above the cutoff | 26 | 83.9 | 14 | 88.9 | 10 | 88.9 | 4 | 88.9 |
| Below the cutoff | 5 | | 112 | | 80 | | 32 | |
| PRELP (cutoff: 0.415) | | | | | | | | |
| Above the cutoff | 28 | 90.3 | 12 | 90.5 | 10 | 88.9 | 2 | 94.4 |
| Below the cutoff | 3 | | 114 | | 80 | | 34 | |
| Combined OMD and PRELP | | | | | | | | |
| Both above the cutoff | 26 | 83.9 | 6 | 95.2 | 5 | 94.4 | 1 | 97.2 |
| At least one below the cutoff | 5 | | 120 | | 85 | | 35 | |
| At least one above the cutoff | 31 | 100 | 20 | 84.1 | 15 | 83.3 | 5 | 86.1 |
| Both below the cutoff | 0 | | 106 | | 75 | | 75 | |
| Renal | | | | | | | | |
| OMD (cutoff: 0.489) | | | | | | | | |
| Above the cutoff | 14 | 87.5 | 12 | 84.8 | 5 | 80.0 | 7 | 87.0 |
| Below the cutoff | 2 | | 67 | | 20 | | 47 | |
| PRELP (cutoff: 0.170) | | | | | | | | |
| Above the cutoff | 12 | 75.0 | 10 | 87.3 | 5 | 80.0 | 5 | 90.7 |
| Below the cutoff | 4 | | 69 | | 20 | | 49 | |
| Combined OMD and PRELP | | | | | | | | |
| Both above the cutoff | 10 | 62.5 | 5 | 93.6 | 2 | 92.0 | 3 | 94.4 |
| At least one below the cutoff | 6 | | 74 | | 23 | | 51 | |
| At least one above the cutoff | 16 | 100 | 17 | 78.4 | 8 | 68.0 | 9 | 83.3 |
| Both below the cutoff | 0 | | 62 | | 17 | | 45 | |

*Early stage: pTa and pT1,
**pT2, pT3 and pT4

TABLE 5
A list of genes regulated by OMD
Genes activated by OMD

| | | | | | |
|---|---|---|---|---|---|
| ACRC | EIF2C2 | LEF1 | PPPIR10 | SOX2 | ZNF655 |
| ANP31A | EIF2S3 | LGALSB | PPP2CB | SOX9 | ZNF791 |
| ARHGEF2 | ENCI | LMNA | PRKAR2A | SP3 | ZRSR1 |
| ASH1L | FAM36A | LOC386167 | PSMD7 | SPTBN1 | ZSWIM6 |
| AURKB | FLJ10769 | LOC644132 | PTP4A1 | SQSTM1 | |
| BACH2 | FOXC1 | LPGAT1 | PTPN1 | SRRM2 | |
| BAT201 | FOXO3 | LSM14B | PURA | SSR3 | |
| BIRC4 | FYN | MALAT1 | QSER1 | STC1 | |
| BRD4 | GATA3 | MCM3APAS | RAB2B | TAOK1 | |
| BU83 | GATC | MDM4 | RAPGEF2 | TBL1X | |
| C10orf46 | GNAS | MGA | RBM42 | TBL1XR1 | |
| C11orf30 | GNG4 | MLL | RBM9 | TGFB1I1 | |
| C16orf52 | GPATCH8 | MRPL38 | RNF12 | THRAP3 | |
| C1orf69 | GTSE1 | MTRF1L | RP11- | TJP2 | |
| C1orf79 | H2AFY | MYH10 | 11C5.2 | TNRC6B | |
| C4orf30 | HIPK1 | MYLIP | RSF1 | TPBG | |
| C7orf29 | HIST1H2AC | NR2F2 | SCHL1 | TROVE2 | |
| CALM1 | HMBOX1 | NUCKS1 | SDCBP | TUBA1A | |
| CASP2 | HNRNPL | ONECUT2 | SEC14L1 | TULP3 | |
| CEP170 | HNRPH1 | OTUD1 | SEC31A | TWIST1 | |
| CHD7 | IL27RA | PABPC1 | SEL1L | UBAP2 | |
| CHKB | IL6ST | PDPK1 | SFPQ | VEZF1 | |
| CUGBP1 | KCNK1 | PHC2 | SFRS6 | VGLL4 | |
| CYCS | KIAA0265 | PHF17 | SFRS7 | WAC | |
| DNAJB1 | KIAA1245 | PIAS1 | SLC16A6 | WDR33 | |
| DNAJC3 | KIAA1333 | PLAGL1 | SLC25A36 | WDR37 | |
| DYNCIH1 | KIAA1641 | PLK2 | SMCHD1 | YTHDF2 | |
| EDEM1 | KLF5 | PMS2L5 | SMTN | ZCCHC10 | |
| EFN82 | KLF6 | POLR1D | SNHG6 | ZNF263 | |
| EGR1 | LDLR | POLR2J3 | SNHG0 | ZNF573 | |
| | | | SON | | |

Genes inhibited by OMD

| | | | | |
|---|---|---|---|---|
| ABHD10 | CSRP2 | MAP3K7 | PNKD | SPARC |
| ADCK2 | DPY19L1 | MAP7 | PNN | SPG21 |
| ADSS | EDNRA | MAPK13 | PPAP2A | SRDSA1 |
| AGA | FAM69A | MBIP | PPM1B | SRI |
| ALDH1A3 | FBXL4 | MBNL1 | PRMT3 | SSFA2 |
| ALDH1B1 | FGFBP1 | MEF2B | PSPH | ST6GALN |
| ANKRA2 | FLJ20489 | METT5D1 | PYGL | AC3 |
| APBB2 | GABPB2 | MOBXL1B | RAB27B | STAU2 |
| ATPBD1C | GGH | MORF4 | RAB7A | STK39 |
| B3GALNT1 | GPX3 | MPHOSP | RAPGEF3 | SUCLG2 |
| BCLAF1 | HEBP1 | M9 | RER1 | SUNO3 |
| C11orf73 | HMOX1 | MPZL2 | RFK | TFRC |
| C19orf42 | HNMT | MRPL44 | SAT1 | TIMM17A |
| C8orf32 | HOXA9 | MTAP | SCOC | TKT |
| CALD1 | HSOL2 | MYO1B | SCRN3 | TMEM138 |
| CALM3 | IMPA2 | NAP1L1 | SEC61A2 | TMEM157 |
| CCDC3 | ISG20L2 | NAT13 | SECTM1 | TMEM192 |
| CD3EAP | ITCH | NFS1 | SELT | TMEM50B |
| CDK8 | ITGA3 | NFVB | SEMA3C | TRIP13 |
| CDK9 | IVD | NIN | SERBP1 | TUBE1 |
| CDS1 | KBTBD7 | NMD3 | SERF1A | UBE2K |
| CHRNA5 | KCTD18 | NT5C3L | SH3YL1 | VAMP3 |
| CISD2 | KLHL8 | OAT | SKP2 | VDAC1 |
| CMAS | KREMEN1 | PAPOLA | SLC1A3 | VEZT |
| CNOT6 | KRTI6 | PCBD1 | SLC39A11 | VGLL3 |
| CNTNAP2 | LACTB2 | PFDN4 | SLC6AB | VWHAH |
| CONMD10 | LOC645619 | P1R | SLC7A11 | ZNF273 |
| CORO1C | LOC648390 | PKNOX1 | SLMO2 | ZNF45 |
| CREB1 | LOC653563 | PLA2G4A | SMAD6 | |
| CREBZF | LRP12 | PLCG2 | SORD | |
| | | PM20D2 | | |

TABLE 6
A list of genes regulated by PRELP

| Genes activated by PRELP | | | | | | Genes inhibited by PRELP |
|---|---|---|---|---|---|---|
| ACADM | CLDND1 | HMGCS1 | NDUFB3 | RAB23 | TNKS2 | ADCY3 |
| ACTR2 | COMMD2 | HMMR | NDUFS1 | RAD21 | TROVE2 | ANKRD52 |
| ADD3 | CSNK1A1 | IL1RAP | NFE2L2 | RBBP9 | TW1STNB | ARID3B |
| AFF4 | CSNK2A1 | IMPACT | NMI | RBMS1 | UBXD2 | BAK1 |
| AMMECR1 | CXorf34 | INSIG2 | NR3C1 | REEP3 | UBXD8 | CALM3 |
| AP1S3 | DC2 | ISCA2 | NSL1 | RHEB | UQCRB | CCNE1 |
| ARL6IP1 | DCK | ITGB8 | NUDT4 | RNF13 | USP1 | COL1A1 |
| ARPP-19 | DCTN4 | KCNK1 | NXT2 | RNF138 | VEZF1 | CRTC3 |
| ASAH1 | DCUN1D4 | KCTD12 | ORMDL1 | RPAP3 | WSB2 | DNMT1 |
| ATP6AP2 | DLAT | KDELC1 | PCMTD1 | RPL22 | YAF2 | FLJ35348 |
| BAX | DLST | KIAA1627 | PCNP | RRAS2 | ZMAT3 | HK1 |
| C10orf104 | DNAJB4 | KLHL28 | PFDN6 | SAMD5 | ZNF706 | IL6R |
| C14orf129 | DPY19L4 | LOC221710 | PGGT1B | SCML1 | | ISG20L2 |
| C15orf29 | EEF1A1 | LOC493869 | PHTF2 | SELT | | KPNB1 |
| C1orf69 | EFCAB7 | LOC550643 | PLK2 | SERP1 | | MFAP2 |
| C4orf29 | EIF2S3 | LOC728866 | PNRC2 | SFRS10 | | MINK1 |
| C5orf22 | ELK3 | LOC730432 | POLR2G | SGCB | | MYH10 |
| C5orf34 | ENOPH1 | LYRM5 | POLR3G | SGPP1 | | NDRG1 |
| C9orf82 | ENPP4 | LYRM7 | PPAT | SGTB | | PHLPPL |
| CASP3 | EXOC5 | LYSMD2 | PPP1CB | SLC16A7 | | PSRC1 |
| CCOC76 | FCF1 | MALAT1 | PPP1R2 | SLC25A36 | | RAC2 |
| CCNE2 | FUSIP1 | MATR3 | PPP2R1B | SLC38A2 | | RHDB |
| CCNG1 | PLSCR1 | MBNL2 | PPTC7 | SLMO2 | | SENP3 |
| CDC2 | GINS1 | MDFIC | PRKAR1A | SMC2 | | SERPINH1 |
| CFL2 | GNAI3 | MIPOL1 | PRPS1 | SPRED1 | | WDR54 |
| CGGBP1 | GNAQ | MLSTD2 | PSPC1 | SSR3 | | |
| CGGBP1 | GNB4 | MOSPD1 | PTP4A2 | SUMO2 | | |
| CHMP4B | GNG12 | MSI2 | PTX3 | SYNJ2BP | | |
| CLASP2 | GPD2 | MTAP | PXMP3 | TAF13 | | |
| CLDN1 | HIST2H2BE | NAT13 | RAB10 | THAP2 | | |

TABLE 7
The KEGG pathway analysis of OMD based on the Affymetrix's microarray data

| Entry ID | Name | Definition | P |
|---|---|---|---|
| OMD | | | |
| hsa04115 | p53 signaling | p53 activation is induced by a number of stress signals, including DNA damage, oxidative | 0.012294 |
| | pathway | stress and activated oncogenes. The p53 protein is employed as a transcriptional activator of | |
| | | p53-regulated genes. Thiese results in three major outputs; cell cycle arrest, cellular | |
| hsa04530 | Tight junction | senescence or apoptosis. | 0.014608 |
| | | Epithelial tight junctions (TJs) are composed of at least three types of transmembrane protein—occludin, | |
| | | claudin and junctional adhesion molecules (JAMs)—and a cyloplasmic ‘plaque’ consisting of | |
| | | many different proteins that form large complexes. | |
| hsa04520 | Adherens junction | Cell-cell adherens junctions (AJs), the most common type of intercellular adhesions, are important for | 0.0160148 |
| | | maintaining tissue architecture and cell polarity and can limit cell movement and proliferation. | |
| hsa04310 | Wnt signaling | Wnt proteins are secreted morphogens that are required for basic developmental processes, such as | 0.0194646 |
| | pathway | cell-fate specification, progenitor-cell proliferation and the control of asymmetric cell division, in many | |
| | | different species and organs. There are at least three different Wnt pathways: the canonical pathway, | |
| | | the planar cell polarity (PCP) pathway and the Wnt/Ca2+ pathway. | |
| hsa04210 | Apoptosis | Apoptosis is a genetically controlled mechanisms of cell death involved in the regulation of tissue | 0.0194646 |
| | | homeostasis. The 2 major pathways of apoptosis are the extrinsic (Fas and other TNFR superfamily | |
| | | members and ligands) and the intrinsic (mitochondria-associated) pathways, both of which are found | |
| | | in the cytoplasm. | |
| hsa05222 | Small cell | Small cell lung carcinoma (SCLC) is a highly aggressive neoplasm, which accounts for approximately | 0.0237281 |
| | lung cancer | 20% of all lung cancer cases. Molecular mechanisms altered in SCLC include induced expression of | |
| | | oncogene, MYC, and loss of tumorsuppressor genes, such as p53, PTEN, RB, and FHIT. The | |
| | | overexpression of MYC proteins in SCLC is largely a result of gene amplification. | |
| PRELP | | | |
| hsa04115 | p53 signaling | p53 activation is induced by a number of stress signals, including DNA damage, oxidative | 4.32 × 10⁻⁵ |
| | pathway | stress and activated oncogenes. The p53 protein is employed as a transcriptional activator of | |
| | | p53-regulated genes. Thiese results in three major outputs; cell cycle arrest, cellular | |
| | | senescence or apoptosis. | |
| hsa04210 | Apoptosis | Apoptosis is a genetically controlled mechanisms of cell death involved in the regulation of tissue | 0.0088029 |
| | | homeostasis. The 2 major pathways of apoptosis are the extrinsic (Fas and other TNFR superfamily | |
| | | members and ligands) and the intrinsic (mitochondria-associated) pathways, both of which are | |
| | | found in the cytoplasm. | |
| hsa04530 | Tight junction | Epithelial tight junctions (TJs) are composed of at least three types of transmembrane protein—occludin, | 0.04122528 |
| | | claudin and junctional adhesion molecules (JAMs)—and a cytoplasmic ‘plaque’ consisting of | |
| | | many different proteins that form large complexes. | |

### REFERENCES

Alimohamad, H., Habijanac, T., Larjava, H., and Hakkinen, L. (2005). Colocalization of the collagen-binding proteoglycans decorin, biglycan, fibromodulin and lumican with different cells in human gingiva. J Periodontal Res 40, 73-86.

Arnaldi, L. A., Borra, R. C., Maciel, R. M., and Cerutti, J. M. (2005). Gene expression profiles reveal that DCN, DIO1, and DIO2 are underexpressed in benign and malignant thyroid tumors. Thyroid 15, 210-221.

Austin, B. A., Coulon, C., Liu, C. Y., Kao, W. W., and Rada, J. A. (2002). Altered collagen fibril formation in the sclera of lumican-deficient mice. Invest Ophthalmol Vis Sci 43, 1695-1701.

Bech-Hansen, N. T., Naylor, M. J., Maybaum, T. A., Sparkes, R. L., Koop, B., Birch, D. G., Bergen, A. A., Prinsen, C. F., Polomeno, R. C., Gal, A., et al. (2000). Mutations in NYX, encoding the leucine-rich proteoglycan nyctalopia, cause X-linked complete congenital stationary night blindness. Nat Genet 26, 319-323.

Bengtsson, E., Aspberg, A, Heinegard, D., Sommarin, Y., and Spillmann, D. (2000). The amino-terminal part of PRELP binds to heparin and heparan sulfate. J Biol Chem 275, 40695-40702.

Bengtsson, E., Morgelin, M., Sasaki, T., Timpl, R., Heinegard, D., and Aspberg, A. (2002). The leucine-rich repeat protein PRELP binds perlecan and collagens and may function as a basement membrane anchor. J Biol Chem 277, 15061-15068.

Bienz, M., and Clevers, H. (2000). Linking colorectal cancer to Wnt signaling. Cell 103, 311-320.

Brown, J. M., and Attardi, L. D. (2005). The role of apoptosis in cancer development and treatment response. Nat Rev Cancer 5, 231-237.

Campo, S., Campo, G. M., Avenoso, A., D′Ascola, A., Musolino, C., Calabro, L., Bellomo, G., Quartarone, E., and Calatroni, A. (2006). Lymphocytes from patients with early stage of B-cell chronic lymphocytic leukaemia and long survival synthesize decorin. Biochimie 88, 1933-1939.

Clevers, H. (2004). Wnt breakers in colon cancer. Cancer Cell 5, 5-6.

Czerniak, B., Chaturvedi, V., Li, L., Hodges, S., Johnston, D., Roy, J. Y., Luthra, R., Logothetis, C., Von Eschenbach, A. C., Grossman, H. B., et al. (1999). Superimposed histologic and genetic mapping of chromosome 9 in progression of human urinary bladder neoplasia: implications for a genetic model of multistep urothelial carcinogenesis and early detection of urinary bladder cancer. Oncogene 18, 1185-1196.

Danielson, K. G., Baribault, H., Holmes, D. F., Graham, H., Kadler, K. E., and Iozzo, R. V. (1997). Targeted disruption of decorin leads to abnormal collagen fibril morphology and skin fragility. J Cell Biol 136, 729-743.

Eissa, S., Kassim, S. K., Labib, R. A., El-Khouly, I. M., Ghaffer, T. M., Sadek, M., Razek, O. A., and El-Ahmady, O. (2005). Detection of bladder carcinoma by combined testing of urine for hyaluronidase and cytokeratin 20 RNAs. Cancer 103, 1356-1362.

Fesik, S. W. (2005). Promoting apoptosis as a strategy for cancer drug discovery. Nat Rev Cancer 5, 876-885.

Giehl, K., and Menke, A. (2008). Microenvironmental regulation of E-cadherin-mediated adherens junctions. Front Biosci 13, 3975-3985.

Grover, J., Lee, E. R., Mounkes, L. C., Stewart, C. L., and Roughley, P. J. (2007). The consequence of PRELP overexpression on skin. Matrix Biol 26, 140-143.

Gudjonsson, S., Isfoss, B. L., Hansson, K., Domanski, A. M., Warenholt, J., Soller, W., Lundberg, L. M., Liedberg, F., Grabe, M., and Mansson, W. (2008). The value of the UroVysion assay for surveillance of non-muscle-invasive bladder cancer. Eur Urol 54, 402-408.

Habuchi, T., Devlin, J., Elder, P. A., and Knowles, M. A. (1995). Detailed deletion mapping of chromosome 9q in bladder cancer: evidence for two tumour suppressor loci. Oncogene 11, 1671-1674.

Hedbom, E., and Heinegard, D. (1993). Binding of fibromodulin and decorin to separate sites on fibrillar collagens. J Biol Chem 268, 27307-27312.

Heinegard, D., Larsson, T., Sommarin, Y., Franzen, A., Paulsson, M., and Hedbom, E. (1986). Two novel matrix proteins isolated from articular cartilage show wide distributions among connective tissues. J Biol Chem 261, 13866-13872.

Hocking, A. M., Shinomura, T., and McQuillan, D. J. (1998). Leucine-rich repeat glycoproteins of the extracellular matrix. Matrix Biol 17, 1-19.

Iozzo, R. V., Chakrani, F., Perrotti, D., McQuillan, D. J., Skorski, T., Calabretta, B., and Eichstetter, I. (1999a). Cooperative action of germ-line mutations in decorin and p53 accelerates lymphoma tumorigenesis. Proc Natl Acad Sci USA 96, 3092-3097.

Iozzo, R. V., Moscatello, D. K., McQuillan, D. J., and Eichstetter, I. (1999b). Decorin is a biological ligand for the epidermal growth factor receptor. J Biol Chem 274, 4489-4492.

Johnstone, R. W., Ruefli, A. A., and Lowe, S. W. (2002). Apoptosis: a link between cancer genetics and chemotherapy. Cell 108, 153-164.

Kizawa, H., Kou, I., Iida, A., Sudo, A., Miyamoto, Y., Fukuda, A., Mabuchi, A., Kotani, A., Kawakami, A., Yamamoto, S., at al. (2005). An aspartic acid repeat polymorphism in asporin inhibits chondrogenesis and increases susceptibility to osteoarthritis. Nat Genet 37, 138-144.

Kuriyama, S., Lupo, G., Ohta, K., Ohnuma, S., Harris, W. A., and Tanaka, H. (2006). Tsukushi controls ectodermal patterning and neural crest specification in Xenopus by direct regulation of BMP4 and X-delta-1 activity. Development 133, 75-88.

Leygue, E., Snell, L., Dotzlaw, H., Hole, K., Hiller-Hitchcock, T., Roughley, P. J., Watson, P. H., and Murphy, L. C. (1998). Expression of lumican in human breast carcinoma. Cancer Res 58, 1348-1352.

Leygue, E., Snell, L., Dotzlaw, H., Troup, S., Hiller-Hitchcock, T., Murphy, L. C., Roughley, P. J., and Watson, P. H. (2000). Lumican and decorin are differentially expressed in human breast carcinoma. J Pathol 192, 313-320.

Li, X., Roginsky, A. B., Ding, X. Z., Woodward, C., Collin, P., Newman, R. A., Bell, R. H., Jr., and Adrian, T. E. (2008). Review of the apoptosis pathways in pancreatic cancer and the anti-apoptotic effects of the novel sea cucumber compound, Frondoside A. Ann N Y Acad Sci 1138, 181-198.

Liotta, L. A. (1986). Tumor invasion and metastases--role of the extracellular matrix: Rhoads Memorial Award lecture. Cancer Res 46, 1-7.

Liu, C. Y., Birk, D. E., Hassell, J. R., Kane, B., and Kao, W. W. (2003). Keratocan-deficient mice display alterations in corneal structure. J Biol Chem 278, 21672-21677.

Lu, Y. P., Ishiwata, T., Kawahara, K., Watanabe, M., Naito, Z., Moriyama, Y., Sugisaki, Y., and Asano, G. (2002). Expression of lumican in human colorectal cancer cells. Pathol Int 52, 519-526.

Morris, S. A., Almeida, A. D., Tanaka, H., Ohta, K., and Ohnuma, S. (2007). Tsukushi modulates Xnr2, FGF and BMP signaling: regulation of Xenopus germ layer formation. PLoS ONE 2, e1004.

Moscatello, D. K., Santra, M., Mann, D. M., McQuillan, D. J., Wong, A. J., and Iozzo, R. V. (1998). Decorin suppresses tumor cell growth by activating the epidermal growth factor receptor. J Clin Invest 101, 406-412.

Naito, Z., Ishiwata, T., Kurban, G., Teduka, K., Kawamoto, Y., Kawahara, K., and Sugisaki, Y. (2002). Expression and accumulation of lumican protein in uterine cervical cancer cells at the periphery of cancer nests. Int J Oncol 20, 943-948.

Nash, M. A., Deavers, M. T., and Freedman, R. S. (2002). The expression of decorin in human ovarian tumors. Clin Cancer Res 8, 1754-1760.

Ohta, K., Kuriyama, S., Okafuji, T., Gejima, R., Ohnuma, S., and Tanaka, H. (2006). Tsukushi cooperates with VG1 to induce primitive streak and Hensen's node formation in the chick embryo. Development 133, 3777-3786.

Ohta, K., Lupo, G., Kuriyama, S., Keynes, R., Holt, C. E., Harris, W. A., Tanaka, H., and Ohnuma, S. (2004). Tsukushi functions as an organizer inducer by inhibition of BMP activity in cooperation with chordin. Dev Cell 7, 347-358.

Olsburgh, J., Harnden, P., Weeks, R., Smith, B., Joyce, A., Hall, G., Poulsom, R., Selby, P., and Southgate, J. (2003). Uroplakin gene expression in normal human tissues and locally advanced bladder cancer. J Pathol 199, 41-49.

Patel, S., Santra, M., McQuillan, D. J., Iozzo, R. V., and Thomas, A. P. (1998). Decorin activates the epidermal growth factor receptor and elevates cytosolic Ca2+ in A431 carcinoma cells. J Biol Chem 273, 3121-3124.

Pellegata, N. S., Dieguez-Lucena, J. L., Joensuu, T., Lau, S., Montgomery, K. T., Krahe, R., Kivela, T., Kucherlapati, R., Forsius, H., and de la Chapelle, A. (2000). Mutations in KERA, encoding keratocan, cause cornea plana. Nat Genet 25, 91-95.

Polakis, P. (2000). Wnt signaling and cancer. Genes Dev 14, 1837-1851.

Pusch, C. M., Zeitz, C., Brandau, O., Pesch, K., Achatz, H., Feil, S., Scharfe, C., Maurer, J., Jacobi, F. K., Pinckers, A., et al. (2000). The complete form of X-linked congenital stationary night blindness is caused by mutations in a gene encoding a leucine-rich repeat protein. Nat Genet 26, 324-327.

Rada, J. A., Cornuet, P. K., and Hassell, J. R. (1993). Regulation of corneal collagen fibrillogenesis in vitro by corneal proteoglycan (lumican and decorin) core proteins. Exp Eye Res 56, 635-648.

Reed, C. C., Waterhouse, A., Kirby, S., Kay, P., Owens, R. T., McQuillan, D. J., and Iozzo, R. V. (2005). Decorin prevents metastatic spreading of breast cancer. Oncogene 24, 1104-1110.

Rehn, A. P., Chalk, A. M., and Wendel, M. (2006). Differential regulation of osteoadherin (OSAD) by TGF-beta1 and BMP-2. Biochem Biophys Res Commun 349, 1057-1064.

Reya, T., and Clevers, H. (2005). Wnt signalling in stem cells and cancer. Nature 434, 843-850.

Santra, M., Skorski, T., Calabretta, B., Lattime, E. C., and Iozzo, R. V. (1995). De novo decorin gene expression suppresses the malignant phenotype in human colon cancer cells. Proc Natl Acad Sci USA 92, 7016-7020.

Schonherr, E., Witsch-Prehm, P., Harrach, B., Robenek, H., Rauterberg, J., and Kresse, H. (1995). Interaction of biglycan with type I collagen. J Biol Chem 270, 2776-2783.

Shimizu-Hirota, R., Sasamura, H., Kuroda, M., Kobayashi, E., Hayashi, M., and Saruta, T. (2004). Extracellular matrix glycoprotein biglycan enhances vascular smooth muscle cell proliferation and migration. Circ Res 94, 1067-1074.

Simoneau, A. R., Spruck, C. H., 3rd, Gonzalez-Zulueta, M., Gonzalgo, M. L., Chan, M. F., Tsai, Y. C., Dean, M., Steven, K., Horn, T., and Jones, P. A. (1996). Evidence for two tumor suppressor loci associated with proximal chromosome 9p to q and distal chromosome 9q in bladder cancer and the initial screening for GAS1 and PTC mutations. Cancer Res 56, 5039-5043.

Simoneau, M., Aboulkassim, T. O., LaRue, H., Rousseau, F., and Fradet, Y. (1999). Four tumor suppressor loci on chromosome 9q in bladder cancer: evidence for two novel candidate regions at 9822.3 and 9q31. Oncogene 18, 157-163.

Sommarin, Y., Wendel, M., Shen, Z., Hellman, U., and Heinegard, D. (1998). Osteoadherin, a cell-binding keratan sulfate proteoglycan in bone, belongs to the family of leucine-rich repeat proteins of the extracellular matrix. J Biol Chem 273, 16723-16729.

Stanford, C. M., Jacobson, P. A., Eanes, E. D., Lembke, L. A., and Midura, R. J. (1995). Rapidly forming apatitic mineral in an osteoblastic cell line (UMR 106-01 BSP). J Biol Chem 270, 9420-9428.

Svensson, L., Aszodi, A., Reinholt, F. P., Fassler, R., Heinegard, D., and Oldberg, A. (1999). Fibromodulin-null mice have abnormal collagen fibrils, tissue organization, and altered lumican deposition in tendon. J Biol Chem 274, 9636-9647.

Taipale, J., and Beachy, P. A. (2001). The Hedgehog and Wnt signalling pathways in cancer. Nature 411, 349-354.

Takeuchi, Y., Kodama, Y., and Matsumoto, T. (1994). Bone matrix decorin binds transforming growth factor-beta and enhances its bioactivity. J Biol Chem 269, 32634-32638.

Tsukita, S., Yamazaki, Y., Katsuno, T., and Tamura, A. (2008). Tight junction-based epithelial microenvironment and cell proliferation. Oncogene 27, 6930-6938.

Vazquez, A., Bond, E. E., Levine, A. J., and Bond, G. L. (2008). The genetics of the p53 pathway, apoptosis and cancer therapy. Nat Rev Drug Discov 7, 979-987.

Vogel, K. G., Paulsson, M., and Heinegard, D. (1984). Specific inhibition of type I and type II collagen fibrillogenesis by the small proteoglycan of tendon. Biochem J 223, 587-597.

Vuillermoz, B., Khoruzhenko, A., D′Onofrio, M. F., Ramont, L., Venteo, L., Perreau, C., Antonicelli, F., Maquart, F. X., and Wegrowski, Y. (2004). The small leucine-rich proteoglycan lumican inhibits melanoma progression. Exp Cell Res 296, 294-306.

Wallard, M. J., Pennington, C. J., Veerakumarasivam, A., Burtt, G., Mills, I. G., Warren, A., Leung, H. Y., Murphy, G., Edwards, D. R., Neal, D. E., et al. (2006). Comprehensive profiling and localisation of the matrix metalloproteinases in urothelial carcinoma. Br J Cancer 94, 569-577.

Weber, C. K., Sommer, G., Michl, P., Fensterer, H., Weimer, M., Gansauge, F., Leder, G., Adler, G., and Gress, T. M. (2001). Biglycan is overexpressed in pancreatic cancer and induces G1-arrest in pancreatic cancer cell lines. Gastroenterology 121, 657-667.

Yu, J., and Zhang, L. (2004). Apoptosis in human cancer cells. Curr Opin Oncol 16, 19-24.

1. A method of discriminating cancer cells from normal cells, which method comprises determining whether a target protein selected from the list consisting of: OMD or a variant thereof; PRELP or a variant thereof, is under-expressed in the cells.

2. A method as claimed in claim 1 wherein the method comprises determining whether both OMD or a variant thereof and PRELP or a variant thereof are under-expressed in the cells.

3. A method as claimed in claim 1 for use in diagnosing, staging or predicting the onset of a cancer in an individual in whom the cells are present or from whom they have been derived.

4. A method for diagnosing, staging or predicting the onset of cancer in a tissue of an individual, which method comprises the steps of:
(a) determining the expression of a target protein or proteins selected from the list consisting of: (i) OMD or a variant thereof; (ii) PRELP or a variant thereof; (iii) both (i) and (ii) in a sample of the tissue from the individual, and
(b) comparing the pattern or level of expression observed in the sample with the pattern or level of expression of the same protein or proteins in or derived from a second clinically normal tissue sample from the same individual or one or more further healthy individuals,
wherein a reduction expression observed in the sample is correlated with the likelihood of the presence of cancer cells in the sample.

5. A method as claimed in claim 1 wherein the pattern or level of expression is assessed
(a) using a nucleic acid sequence encoding all or part of the or each target protein, or a sequence complementary thereto and wherein the level of expression is optionally assessed using an mRNA microarray and RT-PCR, or
(b) by detecting methylation of the promoter region of the gene encoding the or each target protein.

6. (canceled)

7. (canceled)

8. A method as claimed in claim 1 wherein the or each target protein is detected using a recognition compound which is a binding moiety capable of specifically binding the target protein, which binding moiety is optionally linked to a detectable label.

9. A method as claimed in claim 8 wherein the method comprises the steps of (a) obtaining from a patient a tissue sample to be tested for the presence of cancer cells; (b) producing a prepared sample in a sample preparation process; (c) contacting the prepared sample with the recognition compound that reacts with the or each target protein; and (d) detecting binding of the recognition compound to the target protein, if present, in the prepared sample.

10. (canceled)

11. A kit for the diagnosis or prognosis of cancer in a sample, which kit comprises:
(a) a receptacle or other means for receiving a sample to be evaluated, and
(b) a means for specifically detecting the presence and/or quantity in the sample of a target protein or proteins selected from the list consisting of: (i) OMD or a variant thereof; (ii) PRELP or a variant thereof; (iii) both (i) and (ii), and optionally
(c) instructions for performing such an assay.

12. A method for determining the efficacy of a cancer-therapy regime for an individual at one or more time points, said method including the steps of:
(a) determining a baseline value for the expression of a target protein or proteins selected from the list consisting of: (i) OMD or a variant thereof; (ii) PRELP or a variant thereof; (iii) both (i) and (ii) in a cancerous tissue of the individual,
(b) administering a therapeutic drug, and then
(c) redetermining expression levels of the or each target protein within the tissue at one or more instances thereafter,
wherein observed changes in the target protein expression level is correlated with the efficacy of the therapeutic regime.

13. A method of screening for a cancer-therapeutic compound, which method comprises contacting a candidate therapeutic compound with a target protein or proteins selected from the list consisting of: (i) OMD or a variant thereof; (ii) PRELP or a variant thereof and assaying (a) for the presence of a complex between the compound and the target protein, or (b) for the presence of a complex between the target protein and a ligand or binding partner thereof, or (c) assaying the effect of the compound on a biological activity of the target protein.

14. (canceled)

15. A method of producing a model system for screening for a cancer-therapeutic compound, which method comprises:
(a) stably transforming a eukaryotic or prokaryotic host cell with one or more recombinant polynucleotides a target protein or proteins selected from the list consisting of: (i) OMD or a variant thereof; (ii) PRELP or a variant thereof; (iii) both (i) and (ii), or
(b) inactivating within a eukaryotic host cell one or more endogenous genes encoding a target protein or proteins selected from the list consisting of: (i) OMD or a variant thereof; (ii) PRELP or a variant thereof; (iii) both (i) and (ii).

16. (canceled)

17. A transgenic non-human animal, suitable for screening for a cancer-therapeutic compound, which comprises an inactive copy of a gene or genes encoding a target protein or proteins selected from the list consisting of: (i) OMD or a variant thereof; (ii) PRELP or a variant thereof; (iii) both (i) and (ii) target protein.

18. A method of screening for a cancer-therapeutic compound, which method comprises administering a candidate therapeutic compound to an animal as claimed in claim 17 and determining the effect of the therapeutic.

19. A method of screening for a cancer-therapeutic compound, which method comprises:
(a) providing a cell that under-expresses a target protein or proteins selected from the list consisting of: (i) OMD or a variant thereof; (ii) PRELP or a variant thereof; (iii) both (i) and (ii),
(b) adding a candidate therapeutic compound to said cell, and
(c) determining the effect of said compound on the expression or biological activity of said target protein or proteins, and optionally
(d) selecting said compound if it increases the expression or biological activity of said target protein or proteins.

20. A method as claimed in claim 19 which comprises comparing the level of expression or biological activity of the or each protein in the absence of said candidate therapeutic compound to the level of expression or biological activity in the presence of said candidate therapeutic compound.

21. A method as claimed in claim 19 comprising testing for the formation of complexes between a target protein or proteins selected from the list consisting of: (i) OMD or a variant thereof; (ii) PRELP or a variant thereof; (iii) both (i) and (ii) and the compound.

22. A method as claimed in claim 21 comprising testing for the degree to which the formation of a complex between a target protein or proteins selected from the list consisting of: (i) OMD or a variant thereof; (ii) PRELP or a variant thereof; (iii) both (i) and (ii) and a ligand or binding partner is interfered with by the compound.

23. (canceled)

24. (canceled)

25. (canceled)

26. A method of treatment of cancer in a patient in need of the same, which method comprises the step of administering to the patient a therapeutically-effective amount of a compound which increases in vivo expression or activity of a target protein or proteins selected from the list consisting of: (i) OMD or a variant thereof; (ii) PRELP or a variant thereof; (iii) both (i) and (ii).

27. (canceled)

28. A method as claimed in claim 26 wherein the compound is a polynucleotide encoding a target protein or proteins selected from the list consisting of: (i) OMD or a variant thereof; (ii) PRELP or a variant thereof; (iii) both (i) and (ii) and wherein the compound is optionally encoded on a vector.

29. (canceled)

30. A method as claimed in claim 26 wherein the compound interacts with the target protein to increase or augment the biological activity of the target protein.

31. A method or compound as claimed in of claim 26 wherein the compound is used in combination with a DNA damaging reagent which is optionally Mitomycin C.

32. A method as claimed in claim 26 wherein the treatment effects one or more of the following: inhibition of tumourigenesis; cell cycle arrest at G1 phase; inhibition of proliferation; increase in cell death by apoptosis; reduction in anchorage-independent growth or colony-forming ability of cancer cells; increased sensitivity of cancel cells to the therapeutic DNA damaging reagents.

33. A method as claimed in claim 26 wherein the cancer is an epithelial cancer.

34. A use, method or compound as claimed in any one of the claim 26 wherein the cancer is selected from a urological cancer, which is optionally bladder or kidney cancer, or from an other epithelial cancer.

35. A method as claimed in claim 26 wherein cancer are is selected from: lung, breast, stomach, colon, rectum, prostate, utrine cervix, endometrium, ovary, thyroid grand, esophagus, small intestine, and adrenal gland cancers, in which target protein is downregulated.

36. A method as claimed claim 26 wherein the target protein and cancer are selected from: OMD\lung cancer; PRELP\lung cancer; PRELP\Prostate cancer; PRELP\breast cancer.

37. (canceled)


SEQUENCE LISTING (10 sequences)

SEQ ID NO: 1 - protein, 421 aa, Homo sapiens
MGFLSPIYVIFFFFGVKVHCQYETYQWDEDYDQEPDDDYQTGFPFRQNVDYGVPFHQYTLGCVSECFCPTNFPSSMYCDNRKLKTIPNIPMHIQQLYLQFNEIEAVTANSFINATHLKEINLSHNKIKSQKIDYGVFAKLPNLLQLHLEHNNLEEFPFPLPKSLERLLLGYNEISKLQTNAMDGLVNLTMLDLCYNYLHDSLLKDKIFAKMEKLMQLNLCSNRLESMPPGLPSSLMYLSLENNSISSIPEKYFDKLPKLHTLRMSHNKLQDIPYNIFNLPNIVELSVGHNKLKQAFYIPRNLEHLYLQNNEIEKMNLTVMCPSIDPLHYHHLTYIRVDQNKLKEPISSYIFFCFPHIHTIYYGEQRSTNGQTIQLKTQVFRRFPDDDDESEDHDDPDNAHESPEQEGAEGHFDLHYYENQE

SEQ ID NO: 2 - protein, 382 aa, Homo sapiens
MRSPLCWLLPLLILASVAQGQPTRRPRPGTGPGRRPRPRPRPTPSFPQPDEPAEPTDLPPPLPPGPPSIFPDCPRECYCPPDFPSALYCDSRNLRKVPVIPPRIHYLYLQNNFITELPVESFQNATGLRWINLDNNRIRKIDQRVLEKLPGLVFLYMEKNQLEEVPSALPRNLEQLRLSQNHISRIPPGVFSKLENLLLLDLQHNRLSDGVFKPDTFHGLKNLMQLNLAHNILRKMPPRVPTAIHQLYLDSNKIETIPNGYFKSFPNLAFIRLNYNKLTDRGLPKNSFNISNLLVLHLSHNRISSVPAINNRLEHLYLNNNSIEKINGTQICPNDLVAFHDFSSDLENVPHLRYLRLDGNYLKPPIPLDLMMCFRLLQSVVI

SEQ ID NO: 3 - DNA, 20 bp, Artificial sequence
  Synthetic sequence Primer sequence for quantitative RT-PCR
gcaaattccatggcaccgtc

SEQ ID NO: 4 - DNA, 19 bp, Artificial sequence
  Synthetic sequence Primer sequence for quantitative RT-PCR
tcgccccacttgattttgg

SEQ ID NO: 5 - DNA, 20 bp, Artificial sequence
  Synthetic sequence Primer sequence for quantitative RT-PCR
tgggaacaagagggcatctg

SEQ ID NO: 6 - DNA, 22 bp, Artificial sequence
  Synthetic sequence Primer sequence for quantitative RT-PCR
ccaccactgcatcaaattcatg

SEQ ID NO: 7 - DNA, 20 bp, Artificial sequence
  Synthetic sequence Primer sequence for quantitative RT-PCR
gcaaattccatggcaccgtc

SEQ ID NO: 8 - DNA, 19 bp, Artificial sequence
  Synthetic sequence Primer sequence for quantitative RT-PCR
tcgccccacttgattttgg

SEQ ID NO: 9 - DNA, 23 bp, Artificial sequence
  Synthetic sequence Primer sequence for quantitative RT-PCR
ctgtcccacaacaggatcagcag

SEQ ID NO: 10 - DNA, 22 bp, Artificial sequence
  Synthetic sequence Primer sequence for quantitative RT-PCR
caggtccgaggagaagtcatgg